(19) United States
(12) Patent Application Publication
(10) Pub. No.: US 2010/0115640 A1
(43) Pub. Date: May 6, 2010

(54) Methods for Conditional and Inducible Transgene Espression to Direct the Development of Embryonic, Embryonic Stem, Precursor and Induced Pluripotent Stem Cells

(75) Inventors: Chongbei Zhao, Columbia, MO (US); Andras Nagy, Toronto (CA); John K. Critser, Columbia, MO (US)

Correspondence Address: MAGINOT, MOORE & BECK, LLP;CHASE TOWER, 111 MONUMENT CIRCLE, SUITE 3250, INDIANAPOLIS, IN, 46204, US

(73) Assignee: THE CURATORS OF THE UNIVERSITY OF MISSOURI, Columbia, MO (US)

(21) Appl. No.: 12/530,475

(22) PCT Filed: Mar. 7, 2008
(86) PCT No.: PCT/US08/56204

§ 371 Date: Sep. 9, 2009

Related U.S. Application Data
(60) Provisional application No. 60/906,169, filed on Mar. 9, 2007.

(51) Int. Cl. A01K 67/027; C12N 15/11

(52) U.S. Cl. 800/17; 800/21; 800/13; 800/18

(57) ABSTRACT

Methods are disclosed in which the expression of a specific gene, or combinations of genes, is controlled spatially and temporally to develop intra- and interspecies chimeras. A transgenic EC/ES/P/iPS cell line is created which conditionally expresses a suicide or compromiser gene configured to compromise all cell lineages except that corresponding to a target tissue/organ. The EC/ES/P/iPS cell line is injected into donor embryos having a specific target gene deficiency or embryos genetically engineered to be complementary compromised in lineages corresponding to the target tissue/organ cell lineages of the EC/ES/P/iPS line. One or more stimuli is provided to the embryo to activate compromiser genes for ablation of non-target tissues/organs of the EC/ES/P/iPS line and target tissues/organs of the host embryo, resulting in a chimeric animal having target tissues/organs derived from the genotype of the transgenic cell line and all remaining tissues/organs derived from the donor embryo.

### REFERENCE TO RELATED APPLICATION

This application claims priority to co-pending PCT application PCT/U.S.08/056,204, filed on Mar. 7, 2009, the disclosure of which is incorporated herein by reference, which claims priority to U.S. provisional application No. 60/690,169, filed on Mar. 9, 2007, entitled “A Novel Method for Conditional and Inducible Transgene Expression to Specifically and Precisely Direct the Development of Embryonic Cells, Embryonic Stem Cells and Precursor Cells”, the disclosure of which is incorporated herein by reference.

### BACKGROUND

The present disclosure relates to methods to direct the development of embryonic cells, embryonic stem, precursor and induced pluripotent stem (EC/ES/P/iPS) cells to any cell type, tissue or organ system in vitro or in vivo in an exclusive manner, particularly for the creation of chimeras.

The human and mouse genome sequences together created an unprecedented opportunity to develop new, genetically engineered animal models to expedite the development of new treatment modalities to address and relieve human pain and suffering due to diseases. The differentiation program of EC/ES/P/iPS cells is one of the central questions in biology. Furthermore, isolation of tissue-specific stem cells presents a potentially powerful opportunity to develop effective therapeutics to facilitate repair of damaged or diseased organs. The best hope for more rapid discovery of effective prevention and treatment of cancer, cardiovascular disease, diabetes and other catastrophic human diseases, is via enhanced animal models of human health and disease.

Transplantation of organs is a well-known and accepted life-saving procedure for many of these human diseases, such as end-stage kidney, liver, heart and lung diseases. From both a medical and an economic point of view, organ transplantation is often preferable to alternative forms of therapy. But, the insufficient number of donor organs limits the application of this technique and can lead to unnecessary loss of life when other procedures prove ineffectual. Experimental techniques, such as xenotransplantation, have become increasingly more important to develop new methods of creating organ availability.

In past years several kinds of EC/ES/P/iPS cells have been isolated and their differentiation potential has been tested both in vivo and in vitro. However, none of these early studies addressed the “true” physiological fate of such stem cells and progenitor cells as a part of normal development. Several years ago, a novel cell-mapping system was developed which is based on expressing Cre or Flp recombinase in a stem cell or progenitor cell population. See, Dymecki and Tomasiewicz, Dev. Biol. 201:57-65 (1998); Gu et al., Development 129:2447-2457 (2002); and Zinyk et al., Curr. Biol. 8:665-668 (1998). Cre-mediated excision of the “floxed” sequences (i.e., loxP-flanked termination sequences) or Flp-mediated excision of the FRT-flanked sequences in the reporter constructs was shown to result in the permanent expression of the reporter in all the descendant cells. Since Cre or Flp can be introduced into these cells transgenically by using stem cell (or progenitor cell) specific promoter and/or enhancer elements in mice, this strategy permits analysis of the fate of these precursor cells throughout the cells' life in complex organ systems in vivo. A good example of the power of this new recombination-based fate-mapping system is the fate determination of Flk1⁺ cells in mice and proof that Flk1⁺ cells also exhibit a differentiation potential for the other mesodermal lineages than endothelial cells. See, Motoike et al., Genesis 28:75-81 (2003).

Matsumura et al. (2004) reported a new transgenic mouse model with a lineage-specific cell disruption system to express DT which was silent and harmless without the co-expression of Cre recombinase. This mouse provided a model for a variety of studies addressing the consequences of specific cell-type ablations produced by activation of DT expression when it was bred with lineage/cell-specific Cre-expressing mice. See, e.g., Brockschnieder et al., Genesis 44:322-327 (2006) and Kisanuki et al., Developmental Biology 230,230-242 (2001). However, these conditional gene targeting systems have a number of limitations, as they are either spatially controllable or temporally controllable—but not both.

A mutant ligand binding domain of the human estrogen receptor has also been fused to the Cre recombinase by Metzger and Chambon (2001). In transgenic mouse lines produced with this modification, the nuclear localization of the Cre recombinase leads to action that is tamoxifen dependent. These mice have been used to generate cell/organ specific spatio-temporally controlled somatic mutations. The system has been also used in enriching for desired cell types in stem cell differentiation studies.

Two predominant methods have been developed for introducing ES cells into pre-implantation-stage embryos: the so-called injection chimeras and aggregation chimeras. The injection of embryonic cells directly into the cavity of blastocysts is one of the fundamental methods for generating chimeras. ES cells can also be injected into blastocysts, which is probably the most common method for introducing genetic alterations performed in ES cells into mouse by producing germ-line-transmitting chimeras (Bradley et al., Nature 309:255-256 (1984)). Chimeras can also be created by aggregation of embryonic cells with morula-stage embryos. Although ES cells are typically established from the blastocyst stage, they are still capable of integrating one day earlier into the eight-cell-stage embryos. By taking advantage of this property, a relatively simple way of introducing ES cells back into embryonic environment has been developed (Nagy and Rossant, Gene Targeting: A Practical Approach, pp. 177-206 Oxford University Press (1999). Thus, ES cells can also be aggregated with morula-stage embryos to generate chimeras.

### SUMMARY

According to the present method, a novel combination of known genetic tools are used to provide genetically engineered cell, embryo or animal models in which embryonic cells, embryonic stem, precursor and induced pluripotent stem (EC/ES/P/iPS) cells can be precisely directed into desired cell types in intra- or interspecies chimeric composition with differently altered cells in vitro or in vivo. Using this method the expression of a specific gene, or combinations of genes, can be controlled spatially and temporally to develop intra- and interspecies chimeras.

In a preferred embodiment, the method comprises three steps. The first step is to make a transgenic EC/ES/P/iPS cell line which conditionally expresses a suicide or cell progression/existence compromiser gene. Suitable suicide/compromiser genes include Diphtheria Toxin A (DT A), Herpes Simplex Virus-Thymidine Kinase (HSV-TK) or hypoxanthine phosphoribosyltransferase (hprt), although other such genes are contemplated. In the context of the present method, the suicide/compromiser gene is operable to kill target cells or place the target cells at a disadvantage once it is expressed. The time and the type of target cells, i.e., when and where the compromiser gene expression occurs, are controlled by using genetic tools. In certain embodiments, suitable genetic tools include the Cre/loxP, Flp-FRT, and the Tet-inducible recombination systems. In this step, the location of the compromiser gene expression is determined by the gene lineage corresponding to target tissue or organ cells to be derived from the transgenic cell line. Specifically, the compromiser gene is configured to compromise all lineages except that corresponding to the target tissue/organ.

The second step is to aggregate/inject these EC/ES/P/iPS cells into donor embryos. The embryos may have specific gene deficiencies (i.e., knock-out embryos) corresponding to the target lineage. Alternatively, these embryos may be genetically engineered to be complementary compromised in lineages where the EC/ES/P/iPS cells component would be expected to colonize—i.e., the lineage corresponding to the target tissue/organ. The embryo will be a host for the introduced EC/ES/P/iPS cells, establishing the part of the organism where its cells are not compromised. The EC/ES/P/iPS cell contribution may not or may be withdrawn by specific compromiser expression. The complementing part in the organism will be derived exclusively from the introduced EC/ES/P/iPS cells.

The last step of the present embodiment is to apply one or more stimuli to activate the compromiser gene(s) for ablation of undesired tissues/organs of the EC/ES/P/iPS cells and of the host embryo. The stimuli may include exposure of the embryos to a recombination control, such as a particular drug. In a specific example, a suitable drug is a tetracycline.

The present method provides a genetic engineering system for whole organism- or cell-based approaches which can specifically and precisely direct the development of EC/ES/P/iPS cells to desired cell types, tissues or organ systems in vitro or in vivo in an exclusive manner. Using this method, the expression of a specific gene, or combinations of genes, can be controlled spatially and temporally to develop intra- and interspecies in vivo or in vitro chimeric conditions. In these chimeras, a specific cell type, tissue and/or organ system will come exclusively from one component (genotype) and the other cells, tissues and organs are originated from the other component (genotype). For example, this method allows the establishment of a human vasculature (blood vessels) and hematopoietic (blood) system in non-human species such as the mouse or the pig. The method will also enable new approaches to increase the precision of gene therapy methods by differentiating EC/ES/P/iPS cells to specific cell lineages.

According to an alternative embodiment, the method may use genetically modified early cleavage stage embryos or morula embryos (embryonic cells) instead of genetically modified EC/ES/P/iPS cells, in combination with counterpart early cleavage stage or morula embryos instead of blastocysts. These complementary genetically modified cells can then be physically aggregated to produce a viable embryo chimera which can then be transferred to a recipient animal host for gestation and production of live offspring (Nagy et al., Manipulating the Mouse Embryo: A Laboratory Manual, 3d Ed. (2003). A further variation of this method can be to make EC/ES/P/iPS embryonic cell aggregates.

### DESCRIPTION OF THE FIGURES

FIG. 1 is diagram showing the steps of one embodiment of the methods disclosed herein.

FIG. 2 depicts the construction of the LoxP-tet-O-DT-A-pA-loxP [SEQUENCE NO. 1] plasmid used in one embodiment of the method.

FIG. 3 depicts the construction of the HSC-SCL-Cre-ER^{T}-pA plasmid [SEQUENCE NO. 2] used in one embodiment of the method.

FIG. 4 depicts the construction of the Endothelial-SCL-Cre-ER^{T}-pA plasmid [SEQUENCE NO. 3] used in one embodiment of the method.

### DETAILED DESCRIPTION OF THE EMBODIMENTS

Specific language is used to describe several embodiments of this invention to promote an understanding of the invention and its principles. It must be understand that no specific limitation of the scope of this invention is intended by using this specific language. Any alteration and further modification of the described methods or devices, and any application of the principle of this invention are also intended that normally occur to one skilled in this art.

The methods disclosed herein provide genetically engineered animal models that will be extremely helpful to provide new treatment modalities to address human diseases. These animal models may provide a foundation for producing transplantable human organs or tissues, or make such organs and tissues available for drug testing, for instance. In this model, the development of embryonic, embryonic stem, precursor and induced pluripotent stem (EC/ES/P/iPS) cells in an in vitro and in vivo chimeric organism can be precisely directed to any cell type, tissue or organ system in an exclusive manner. In one example, this method allows the establishment of a human vascular endothelium (blood vessels) and hematopoietic (blood) system in non-human species such as the mouse or the pig.

The present method first makes use of cell depletion due to compromiser genes. Examples of suitable compromiser genes include: diphtheria toxin A (DT A), as demonstrated by Ivanova et al., in the article “In vivo genetic ablation by Cre-mediated expression of diphtheria toxin fragment A”, Genesis 43:129-135 (2005), the disclosure of which is incorporated herein by reference; or Herpes Simplex Virus-Thymidine Kinase (HSV-TK). The present method further makes use of certain genetic tools such as: Cre/LoxP as disclosed by Sauer et al., in U.S. Pat. No. 4,959,317, the disclosure of which is incorporated herein by reference; or Flp/FRT, as described by Wahl et al., in U.S. Pat. No. 5,654,182, the disclosure of which is also incorporated herein by reference. These tools further include recombination systems, such as the recombination system demonstrated by Nagy in the article “Cre recombinase: the universal reagent for genome tailoring”, Genesis 26:99-109 (2000), the disclosure of which is incorporated herein by reference.

In a final step of the method, inducible gene expression system are implemented, such as the tetracycline inducible system described by Bujard et al., in U.S. Pat. No. 5,814,618, the disclosure of which is incorporated herein by reference; or by Belteki et al., in the article “Conditional and inducible transgene expression in mice through the combinatorial use of Cre-mediated recombination and tetracycline induction”, Nucleic Acids Research 33, No. 5 (2005), the disclosure of which is also incorporated herein by reference. Using a combination of these tools, the present method contemplates precisely spatially and temporally controlling the expression of cell-specific genes (compromiser) during the development or differentiation processes.

By way of example the method disclosed herein allows the establishment of a human vasculature (blood vessels) and hematopoietic (blood) system in a non-human species such as the mouse or the pig. First, a novel mouse embryonic stem cell (ESC) line will be created which combines all the required genetic tools and inducible systems. In this ESC line, tetracycline inducible compromiser genes are flanked by recombinase attachment sites, such as loxP sites, so that recombinase will delete the compromiser in the lineage of its specificity of expression. A novel transgenic mice line will be produced which is specific gene deficient or in which the inducible compromiser has exactly complementing specificity of expression. This can be achieved by making the reverse tetracycline transactivator recombinase excision conditional, as described by Gossen et al., in the article “Transcriptional activation by tetracyclines in mammalian cells”, Science 23 Jun. 1995 268:1766-1769 (1995), the disclosure of which is incorporated herein by reference.

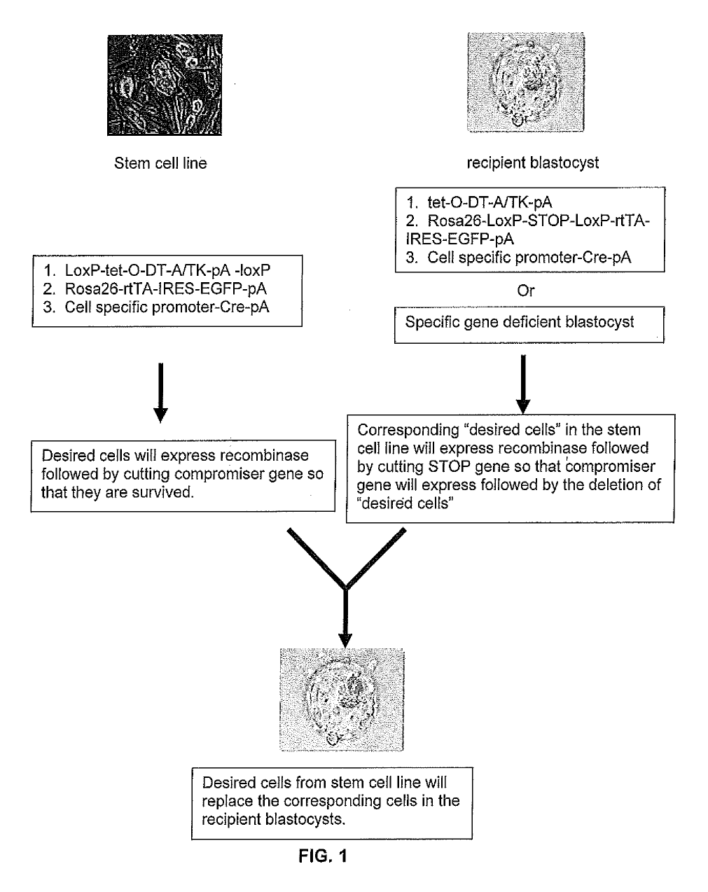

Chimeras will be formed between these ESC and embryos and the chimeras will be incubated or will be transferred to pseudo-pregnant recipients, such as in a manner described by Voncken in “Genetic modification of the mouse: Transgenic mouse—methods and protocols”, Methods in Molecular Biology, Volume 209 (2003), the disclosure of which is incorporated herein by reference. By administering inducible drugs to the recipient mice, such as doxycycline (a derivative of tetracycline), at specific times in development of the embryo, the expression of recombinase and compromiser genes in the chimeric embryos/fetuses will be regulated. This method will be used to establish chimeras in which, by way of non-limiting example, there is a vascular endothelium and hematopoietic system from one genotype (i.e., from the donor ESCs) with all other tissues from another genotype (i.e., from the recipient), as depicted in the diagram of FIG. 1.

### EXAMPLES

The following examples will serve to illustrate the application of the methods described herein.

### Example 1

### Spatial and Temporal Regulation of Endothelial and Hematopoietic-Specific Gene Expression and its Application in Mouse Esc-Mouse Chimeras

FLK1 is a receptor tyrosine kinase and the main signaling receptor for Vascular Endothelial Growth Factor-A (VAGF-A) during embryonic development and adult neovascularization. (Millauer et al., Cell 72:835-846 (1993), Nature 367:576-579 (1994); Goede et al., Lab Invest. 78:1385-1394 (1998)). Analysis of FLK1 knock-out mice by Shalaby et al., (Nature 376:62-66 (1995), Cell 89:981-990 (1997)) revealed a central role of FLK1 in hematopoietic and endothelial development. Licht and co-workers created a novel transgenic mouse line of FLK1-Cre and then cross-bred with the LacZ report mouse line. (Licht et al., Development Dynamics 229:312-318 (2003)). They detected strong, reproducible LacZ staining primarily in the endothelium of blood vessels, but also in circulating blood cells. An almost complete vascular staining was found at mid-gestation and persisted in all organ systems examined in adult mice.

The stem cell leukemia gene (SCL) encodes a basic helix-loop-helix transcription factor with a pivotal role in both hematopoiesis and endothelial development. During mouse development, SCL is first expressed in extra-embryonic mesoderm, and is required for the generation of all hematopoietic lineages and normal yolk sac angiogenesis. SCL deficient embryos lacked yolk sac hematopoiesis and large vitelline vessels although endothelial capillary spaces were present in SCL-l-yolk sac, as demonstrated by Lorraine, et al. (Proc. Natl. Acad. Sci. USA, VOL. 92, pp. 7075-7079), and substantiated by Shivdasani et al. (Nature (London) 373:432-434 (1995)). To address that the lineage relationship between embryonic and adult hematopoietic stem cells (HSC) in the mouse exists, Joachim et al. (Blood 1 April, Vol. 105, No. 7 (2005)) generated transgenic mice which expressed the tamoxifen inducible Cre-ER^{T} recombinase under the control of the stem-cell enhancer of SCL locus (HSC-SCL-Cre-ER^{T}-pA) (Sanchez, et al. Development 126:3891-3904 (1999), Development 128:4815-4827 (2001); Gottgens, et al., EMBO J 21:3039-3050 (2002)). and proved that tamoxifen-dependent recombination occurred in more than 90% of adult long-term HSCs. This experiment was a clear demonstration of successful inducible genetic manipulation of HSCs in vivo.

The FLK1 and SCL play crucial roles in the establishment of hematopoietic and endothelial cell lineages in mice. Changwon et al. (Development and Disease 131:2749-2762 (2004)) have previously used an in vitro differentiation model of embryonic stem (ES) cells and demonstrated that hematopoietic and endothelial cells develop via sequentially generated FLK1⁺ and SCL⁺ cells.

Where the Cre recombinase expression specificity is determined by the endothelial and blood precursor specific promoters, cells derived from the ESC component of the chimeras and differentiated into all non-endothelium and non-hematopoietic (i.e., non-target) lineages will be eliminated by inducing the expression of compromiser genes. At the same time, cells derived from the donor ESC line that developed into target endothelium and hematopoietic lineages will not express the compromiser genes and therefore will survive. Reciprocally, the cells derived from embryo component of the chimeras and differentiated into endothelium and hematopoietic lineages will be eliminated by inducing the expression of compromiser genes. Conversely, cells derived from the embryo component and developed into all non-endothelium and non-hematopoietic lineages will not express the compromiser genes and therefore will survive. As a result, in these chimeras the ESC and embryo components will complement each other; the endothelium and hematopoietic cells will be built from the ESC component, while the embryo component will provide the remaining cells/structure of the chimera.

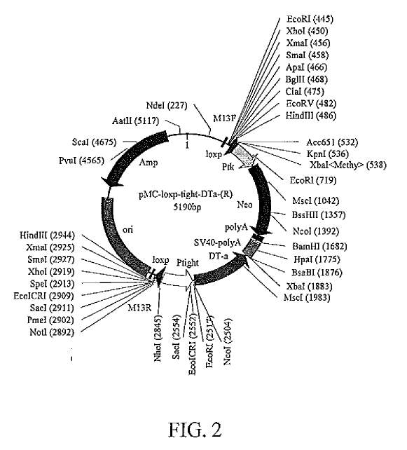

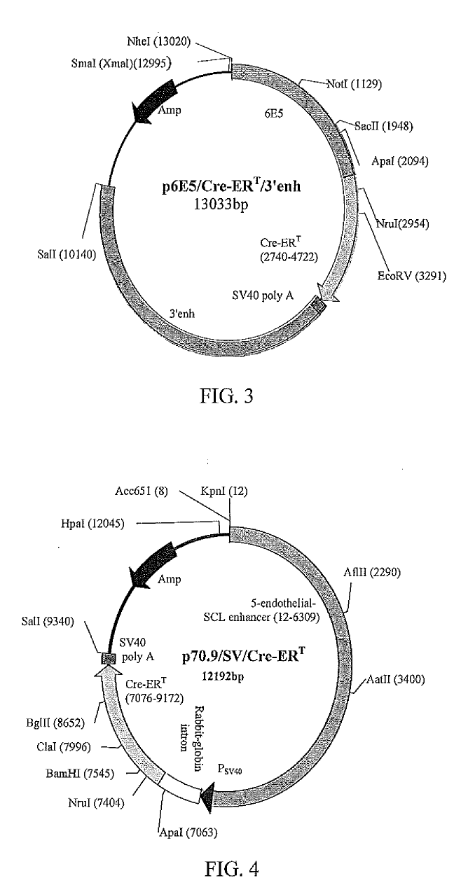

Applying the present method to this example, a new mouse ESC line will be created which contains LoxP-tet-O-DT-A-pA-loxP (FIG. 2 and SEQUENCE NO. 1), Rosa26-rtTA-IRES-EGFP-pA (Enhanced Green Fluorescent Protein, as disclosed in U.S. Pat. No. 5,625,048, the disclosure of which is incorporated herein by reference), FLK1-Cre-pA and HSC-SCL-Cre-ER^{T}-pA (FIG. 3 and SEQUENCE NO. 2). Mouse SCL−/− recipient blastocysts will be created by breeding SCL−/+mice or mouse recipient blastocysts will be created which contain tet-O-DT-A-pA, Rosa26-LoxP-STOP-LoxP-rtTA-IRES-EGFP-pA, FLK1-Cre-pA and HSC-SCL-Cre-ER^{T}-pA. The new ESC line will then be injected into recipient blastocysts and embryo transfer performed according to suitable techniques, such as that described by Voncken.

A Tet-On and Cre-LoxP system will be combined to regulate specific genes' expression by introducing a recombination control drug, such as tetracycline, into the host embryos. In the stem cells system, when endothelial/hematopoietic cell-specific promoters of FLK1 and SCL express, Cre recombinase will be expressed followed by excision of LoxP recognition sites which contain DT-A. Meanwhile, the lineages other than the target endothelial and hematopoietic lineage will express DT-A which kills the cells. In the recipient blastocysts system, SCL−/− blastocysts are hematopoietic and endothelial cells deficient which will be rescued by stem cells because in the blastocysts, this gene regulatory program is working in an opposite way relative to that in stem cell line. When FLK1 and SCL are expressed, Cre recombinase is expressed followed by excision of STOP gene which stops expression of rtTA. After this stop is removed, the tet-O system is activated and DT-A will be expressed. The result is that the recipient blastocysts will be hematopoietic and endothelial deficient and will be “rescued” by the cells coming from donor stem cell system.

By phenotyping the resulting chimeras to confirm different genotypes of the vascular endothelium and hematopoietic system vs. other tissues, it will be possible to identify if the endothelial and hematopoietic cells differentiated from the ESC line rescued the target lineage of the recipient blastocysts.

Alternatively, a stem cell line will be made with constructs of SCL-Cre and Rosa 26-loxP-TK-loxP. By injecting this cell line into SCL −/− embryos, the hematopoietic and endothelial system in the SCL −/− embryos will be replaced with the corresponding system from the stem cell line.

### Example 2

### Spatial and Temporal Regulation of Endothelial and Hematopoietic-Specific Gene Expression and its Application in Human ESC-Mouse Chimeras

The highly conserved basic helix-loop-helix (bHLH) transcription factor SCL has been shown in mice and zebrafish to play a crucial role in patterning of mesoderm into blood and endothelial lineages by regulating the development of the hemangioblast. See, for instance, Labastie et al., Blood 92:3624-3635 (1998) and Lorraine et al., EMBO J. 15:4123-4129 (1996), Proc. Natl. Acad. Sci. USA Vol. 92, pp. 7075-7079 (1995). To address the role SCL plays in normal human developmental hematopoiesis, Elias's work (Elias, et. al, Blood 106:860-870 (2005)) provide insight into the role that key hematopoietic genes may play in human embryonic development. Elias' data revealed that SCL was the first and most dramatically up-regulated gene coinciding with emergence of primitive hematopoiesis and was expressed abundantly in all hematopoietic colonies.

The SCL gene is expressed in a subset of blood cells, endothelial cells, and specific regions of the brain and spinal cord. This pattern of expression is highly conserved throughout vertebrate evolution from zebrafish to mammals. Systematic analysis of the murine SCL locus has identified a series of independent enhancers, each of which directs reporter gene expression to a subdomain of the normal SCL expression pattern. Of particular interest is a 3′enhancer that directs expression to blood and endothelial progenitors throughout ontogeny. See, Sanchez, et al., Development 126:3891-3904 (1999). Joachim, et al. (Blood 104:1769-1777 (2004)) generated endothelial-SCL-Cre-ER^{T} mice using inducible Cre recombinase driven by the 5-endothelial enhancer of the SCL locus. By intercrossing with Cre reporter mice, Joachim found Cre-mediated recombination in almost all endothelial cells of the developing vasculature.

Combining all this information, mouse-human chimeras can be made using the methods described in Example 1. A new human ESC line will be created which contains LoxP-tet-O-DT-A-pA-loxP (FIG. 2 and SEQUENCE NO. 1), Rosa26-rtTA-IRES-EGFP-pA and SCL-Cre-pA (FIG. 3 and SEQUENCE NO. 3). Meanwhile, mouse SCL−/− recipient blastocysts will be created, or alternatively recipient blastocysts will be created which contain tet-O-DT-A-pA, Rosa26-LoxP-STOP-LoxP-rtTA-IRES-EGFP-pA, and SCL-Cre-pA. The new ESC line will be injected into recipient blastocysts and embryo transfer will be performed.

The site-specific recombination systems will be activated at a pre-determined time in the development of the embryo by administration of a recombination control, such as the drug doxycycline. Expression of the suicide/compromiser genes in the ESC line and the donor embryo will result in reciprocal ablation of the non-target cells in the ESC line and the target cells in the donor embryo. The ESC line will thus provide the target cells, in this case vascular endothelium and hematopoietic tissues, for the developing chimeric mouse. The resulting chimeras can be phenotyped to confirm different genotypes of the vascular endothelium and hematopoietic system vs. other tissues. In these chimeras, the endothelial and hematopoietic cells will be human genome background while all the other tissues and organs will be mouse genome background.

### Example 3

### Spatial and Temporal Regulation of Endothelial and Hematopoietic-Specific Gene Expression and its Application in Human ESC-Pig Chimeras

The chronic shortage of human organs, tissues and cells for transplantation has inspired research on the possibility of using animal donor tissue instead of human donor tissue. Transplantation over a species barrier is associated with rejections which are difficult to control. Therefore, it is has been proposed that successful pig to human xenotransplantation requires donor pigs to be genetically modified. See, Prather et al. Theriogenology 59:115-123 (2003); and Kolber-Simonds et al. PNAS 101:7335-7340 (2004). Vascular endothelium is the most immediate barrier between the xenogeneic donor organ and host immune and non-immune defense systems. Thus, these cells are the prime targets for such genetic modifications.

Godwin et al. (Xenotransplantation 13(6):514-521 (2006)) cloned and characterized the regulatory elements of the pig intercellular adhesion molecule-2 (ICAM-2) gene. They observed that a 0.90-kb pig ICAM-2 promoter fragment had strong activity in pig endothelial cells but not in non-endothelial cells. Deletion analysis revealed that the majority of promoter activity was specified by a 0.48-kb sub-fragment with significant homology to the human ICAM-2 promoter. Significant enhancer activity was identified within the first intron of the pig ICAM-2 gene.

The Tie2 promoter and intron/enhancer element has been previously shown to drive reporter genes in vitro and in vivo. Inclusion of a Tie2 intronic enhancer element in conjunction with the Tie2 promoter in Tie2-βgal transgenic mice has resulted in expression in embryonic and adult endothelium as expected, as reported by Schlaeger et al. (Proc. Nat. Acad. Sci. USA 94:3058-3063 (1997)). This same type of promoter-element transgene design was used to generate Tie2-Cre and Tie2-GFP transgenic mice, and Tie2-GFP transgenic Zebrafish (Constien et al. Genesis 30:36-44 (2001); Motoike et al. Genesis 28:75-81 (2000)). Hao et al. (Transgenic Research DI 10.1007/s11248-00609020-8 (2006)) have generated transgenic Yucatan pigs that express the eNOS cDNA under the Tie2 endothelial-specific promoter and Tie2 intron/enhancer element and have demonstrated a similar expression profile in the endothelial compartment in the Tie2-eNOS transgenic swine by immunohistochemistry.

So far, there is no specific gene known which will regulate the differentiation of hematopoietic stem cells from embryonic stem cells in pig. But, it is known that the pattern of SCL gene expression is highly conserved throughout vertebrate evolution from zebrafish to mammals. Thus a promoter of SCL gene can be used to regulate the hematopoietic development in swine.

Consequently, pig-human chimeras can be made using the methods described in Example 1. A new human ESC line will be created which contains LoxP-tet-O-DT-A-pA-loxP, Rosa26-rtTA-IRES-EGFP-pA, SCL-Cre-pA and ICAM-Cre-pA/Tie2-Cre-pA. Concurrently, pig SCL−/− recipient blastocysts will be created or alternatively recipient blastocysts will be created which contain tet-O-DT-A-pA, Rosa26-LoxP-STOP-LoxP-rtTA-IRES-EGFP-pA, SCL-Cre-pA and ICAM-Cre-pA/Tie2-Cre-pA. The new ESC line will be injected into recipient blastocysts and embryo transfer will be performed.

The site-specific recombination systems will be activated at a pre-determined time in the development of the embryo by administration of a recombination control, such as the drug doxycycline. Expression of the suicide/compromiser genes in the ESC line and the donor embryo will result in reciprocal ablation of the non-target cells in the ESC line and the target cells in the donor embryo. The ESC line will thus provide the target cells, in this case vascular endothelium and hematopoietic tissues, for the developing chimeric pig. Finally, the resulting chimeras will be phenotyped to confirm different genotypes of the vascular endothelium and hematopoietic system vs. other tissues. In these chimeras, the endothelial and hematopoietic cells will be human genome background while all the other tissues and organs will be pig genome background.

### Example 4

### Spatial and Temporal Regulation of any Organ/Tissue-Specific Gene Expression and its Application in Chimeras

Based on the method described above, chimeras of any species can be for which EC/ES/P/iPS cells are available and for which the specific promoter/enhancer required to genetically control the chimeric characteristics is known. These chimeras can be created at various stages of embryonic development. In the present example this process can be used at a point in development in the formation of the initial three (triploblastic) tissue layers, namely the endoderm, ectoderm and mesoderm. In this example, inducing chimerism in one of these tissue lineages will result in all subsequent cells, tissues and organs that are derived from a different genotype.

For example, using this method, a pig with a human endoderm lineage can be made. In one specific embodiment, when a specific promoter/enhancer for endoderm is observed which might be called END, the new ESC line of any kind of background would be created which contains LoxP-tet-O-DT-A-pA-loxP, Rosa26-rtTA-IRES-EGFP-pA and END-Cre-pA. Meanwhile, END−/− recipient blastocysts would be created or alternatively blastocysts of any kind of background would be created which contain tet-O-DT-A-pA, Rosa26-LoxP-STOP-LoxP-rtTA-IRES-EGFP-pA, and END-Cre-pA. The new ESC line would be injected into recipient blastocysts and embryo transfer performed.

The site-specific recombination systems will be activated at a pre-determined time in the development of the embryo by administration of a recombination control, such as the drug doxycycline. Expression of the suicide/compromiser genes in the ESC line and the donor embryo will result in reciprocal ablation of the non-target cells in the ESC line and the target cells in the donor embryo. The ESC line will thus provide the target cells for the developing chimeric animal. Finally, the resulting chimeras would be phenotyped to confirm different genotypes of all the tissues/organs coming from endoderm layers vs. other tissues/organs. In these chimeras, the cells coming from endoderm layer will be one genome background and all the other tissues and organs will be the other genome background.

### Example 5

### Spatial and Temporal Regulation of Specific Gene Expression and its Application in Embryonic Cell Derived Chimeras In Vitro

Examples 1-4 described above contemplate spatial and temporal regulation of specific gene expression in vivo. In the present example, this method will be used in vitro as well. As in the prior examples, a new ESC line or ECs will be created which contains three transgenes: (1) loxP-tet-O-DT-A-pA-loxP, (2) Rosa26-rtTA-IRES-EGFP-pA, (3) FLK1-Cre-pA/HSC-SCL-Cre-ERT-pA. Instead of blastocysts injection, chimeras will be made by ES cell-diploid/tetraploid embryo aggregation and injection.

The new ESC line will be created to contain LoxP-tet-O-DT-A-pA-loxP, Rosa26-rtTA-IRES-EGFP-pA and END-Cre-pA. Meanwhile, END−/− recipient diploid embryos would be created or alternatively embryos of any kind of background would be created which contain tet-O-DT-A-pA, Rosa26-LoxP-STOP-LoxP-rtTA-IRES-EGFP-pA, and END-Cre-pA. ESC line will be aggregated with recipient embryos and cultured in vitro. Before embryo transfer, inducible drugs will be administered which will result in embryo chimeras having endoderm lineage that comes from the ESC line while the ectoderm and mesoderm lineages come from the recipient blastocysts.

The resulting chimeras would be phenotyped in vitro to confirm different genotypes of all the tissues/organs coming from endoderm layers vs. other tissues/organs. In these chimeras, the cells coming from endoderm layer will be one genome background and all the other tissues and organs will be the other genome background.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

| SEQUENCE NO. 1 | |
|---|---|
| Restriction analysis on pMC-loxp-tight-DTa-(R).seq | |
| Methylation: dam-No dsm-No | |
| Enzymes with >3 sites are not shown | |
| Screened with 51 enzymes, 64 sites found | |
| AstII GACGT/C 1: 5117 | |
| Acc651 G/GTACC 1: 532 | |
| ApaI GGGCC/C 1: 466 | |
| ApaLI G/TGCAC 3: 220, 3616, 4862 | |
| BamHI G/GATCC1: 1682 | |
| BglI GCCNNNN/NCCG 3: 294, 461, 4315 | |
| BglII A/GATCT 1: 468 | |
| BsaBI GATNN/NNATC 1: 1876 | |
| BssHII G/CGCGC 1: 1357 | |
| ClaI AT/CGAT 1: 475 | |
| EcoICRI GAG/CTC 2: 2552, 2909 | |
| EcoRI G/AATTC 3: 445, 719, 2517 | |
| EcoRV GAT/ATC 1: 482 | |
| HindIII A/AGCTT 2: 486, 2944 | |
| HpaI GTT/AAC 1: 1775 | |
| KpnI GGTAC/C 1: 536 | |
| loxp 2: 514, 2867 | |
| MscI TGG/CCA 2: 1042, 1983 | |
| NcoI C/CATGG 2: 1392, 2504 | |
| NdeI CA/TATG 1: 227 | |
| NheI G/CTAGC 1: 2845 | |
| NotI GC/GGCCGC 1: 2892 | |
| PmeI GTTT/AAAC 1: 2902 | |
| PstI CTGACA/G 3: 816, 1013, 2940 | |
| PvuI CGAT/CG 2: 322, 4565 | |
| PvuII CAG/CTG 3: 351, 1066, 3127 | |
| SacI GAGCT/C 2: 2554, 2911 | |
| ScaI AGT/ACT1: 4675 | |
| SmaI CCC/GGG 2: 458, 2927 | |
| SpeI A/CTAGT 1: 2913 | |
| StuI AGG/CCT 3: 591, 662, 2567 | |
| XbaI T/CTAGA 2: 538, 1883 | |
| XbaI <Methy> T/CTAGATC 1: 538 | |
| XhoI C/TCGAG 2: 450, 2919 | |
| XmaI C/CCGGG 2: 456, 2925 | |
| XmnI GAANN/NNTTC 2: 2881, 4794 | |
| Non Cut Enzymes | |
| Acc65I<Methy>AflII ApaI<Methy> BstEII BstXI ClaI<Methy> | |
| I-PpoI I-SceI MscI<Mety> NruI NruI<Methy> SacII | |
| SalI | |
| ORIGIN | |
| 1 | CTGCCTCGCG CGTTTCGGTG ATGACGGTGA AAACCTCTGA CACATGCAGC TCCCGGAGAC |
| 61 | GGTCACAGCT TGTCTGTAAG CGGAGCCGGG AGCAGACAAG CCCGTCAGGG CGCGTCAGCG |
| 121 | GGTGTTGGCG GGTGTCGGGG CGCAGCCATG ACCCAGTCAC GTAGCGATAG CGGAGTGTAC |
| 181 | TGGCTTAACT ATGCGGCATC AGAGCAGATT GTACTGAGAG TGCACCATAT GCGGTGTGAA |
| 241 | ATACCGCACA GATGCGTAAG GAGAAAATAC CGCATCAGGC GCCATTCGCC ATTCAGGCTA |
| 301 | CGCAACTGTT GGGAAGGGCG ATCGGTGCGG GCCTCTTCGC TATTACGCCA GCTGGCGAAG |
| 361 | GGGGGATGTG CTGCAAGGCG ATTAAGTTGG GTAACGCCAG GGTTTTCCCA GTCACGACGT |
| 421 | TGTAAAACGA CGGCCAGGGC CAGTGAATTC TCGAGCCCGG GGGGCCCAGA TCTATCGATG |
| 481 | ATATCAAGCT TGGTACTATA ACTTCGTATA GTATACATTA TACGAAGTTA TGGTACCTCT |
| 541 | AGATCGACAG TGTGGTTTTG CAAGAGGAAG CAAAAAGCCT CTCCACCCAG GCCTGGAATG |
| 601 | TTTCCACCCA ATGTCGAGCA GTGTGGTTTT GCAAGAGGAA GCAAAAAGCC TCTCCACCCA |
| 661 | GGCCTGGAAT GTTTCCACCC AATGTCGAGC AAACCCCGCC CAGCGTCTTG TCATTGGCGA |
| 721 | ATTCGAACAC GCAGATGCAG TCGGGGCGGC GCGGTCCCAG GTCCACTTCG CATATTAAGG |
| 781 | TGACGCGTGT GGCCTCGAAC ACCGAGCGAC CCTGCAGCCA ATATGGGATC GGCCATTGAA |
| 841 | CAAGATGGAT TGCACGCAGG TTCTCCGGCC GCTTGGGTGG AGAGGCTATT CGGCTATGAC |
| 901 | TGGGCACAAC AGACAATCGG CTGCTCTGAT GCCGCCGTGT TCCGGCTGTC AGCGCAGGGG |
| 961 | CGCCCGGTTC TTTTTGTCAA GACCGACCTG TCCGGTGCCC TGAATGAACT GCAGGACGAG |
| 1021 | GCAGCGCGGC TATCGTGGCT GGCCACGACG GGCGTTCCTT GCGCAGCTGT GCTCGACGTT |
| 1081 | GTCACTGAAG CGGGAAGGGA CTGGCTGCTA TTGGGCGAAG TGCCGGGGCA GGATCTCCTG |
| 1141 | TCATCTCACC TTGCTCCTGC CGAGAAAGTA TCCATCATGG CTGATGCAAT GCGGCGGCTG |
| 1201 | CATACGCTTG ATCCGGCTAC CTGCCCATTC GACCACCAAG CGAAACATCG CATCGAGCGA |
| 1261 | GCACGTACTC GGATGGAAGC CGGTCTTGTC GATCAGGATG ATCTGGACGA AGAGCATCAG |
| 1321 | GGGCTCGCGC CAGCCGAACT GTTCGCCAGG CTCAAGGCGC GCATGCCCGA CGGCGAGGAT |
| 1381 | CTCGTCGTGA CCCATGGCGA TGCCTGCTTG CCGAATATCA TGGTGGAAAA TGGCCGCTTT |
| 1441 | TCTGGATTCA TCGACTGTGG CCGGCTGGGT GTGGCGGACC GCTATCAGGA CATAGCGTTG |
| 1501 | GCTACCCGTG ATATTGCTGA AGAGCTTGGC GGCGAATGGG CTGACCGCTT CCTCGTGCTT |
| 1561 | TACGGTATCG CCGCTCCCGA TTCGCAGCGC ATCGCCTTCT ATCGCCTTCT TGACGAGTTC |
| 1621 | TTCTGAGGGG ATCGGCAATA AAAAGACAGA ATAAAACGCA CGGGTGTTGG GTCGTTTGTT |
| 1681 | CGGATCCGTC GAGGCAGTGA AAAAAATGCT TTATTTGTGA AATTTGTGAT GCTATTGCTT |
| 1741 | TATTTGTAAC CATTATAAGC TGCAATAAAC AAGTTAACAA CAACAATTGC ATTCATTTTA |
| 1801 | TGTTTCAGGT TCAGGGGGAG GTGTGGGAGG TTTTTTAAAG CAAGTAAAAC CTCTACAAAT |
| 1861 | GTGGTATGGC TGATTATGAT CCTCTAGACT CACACCACAG AAGTAAGGTT TCCTTCACAA |
| 1921 | AGAGATCGCC TGACACGATT TCCTGCACAG GCTTGAGCCA TATACTCATA CATCGCATCT |
| 1981 | TGGCCACGTT TTCCACGGGT TTCAAAATTA ATCTCAAGTT CTACGCTTAA CGCTTTCGCC |
| 2041 | TGTTCCCAGT TATTAATATA TTCAACGCTA GAACTCCCCT CAGCGAAGGG AAGGCTGAGC |
| 2101 | ACTACACGCG AAGCACCATC ACCGAACCTT TTGATAAACT CTTCCGTTCC GACTTGCTCC |
| 2161 | ATCAACGGTT CAGTGAGACT TAAACCTAAC TCTTTCTTAA TAGTTTCGGC ATTATCCACT |
| 2221 | TTTAGTGCGA GAACCTTCGT CAGTCCTGGA TACGTCACTT TGACCACGCC TCCAGCTTTT |
| 2281 | CCAGAGAGCG GGTTTTCATT ATCTACAGAG TATCCCGCAG CGTCGTATTT ATTGTCGGTA |
| 2341 | CTATAAAACC CTTTCCAATC ATCGTCATAA TTTCCTTGTG TACCAGATTT TGGCTTTTGT |
| 2401 | ATACCTTTTT GAATGGAATC TACATAACCA GGTTTAGTCC CGTGGTACGA AGAAAAGTTT |
| 2461 | TCCATCACAA AAGATTTAGA AGAATCAACA ACATCATCAG GGTCCATGGT GGCGGCGAAT |
| 2521 | TCTCCAGGCG ATCTGACGGT TCACTAAACG AGCTCTGCTT ATATAGGCCT CCCACCGTAC |
| 2581 | ACGCCTACCT CGACATACGT TCTCTATCAC TGATAGGGAG TAAACTCGAC ATACGTTCTC |
| 2641 | TATCACTGAT AGGGATAAAC TCGACATACG TTCTCTATCA CTGATAGGGA GTAAACTCGA |
| 2701 | CATACGTTCT CTATCACTGA TAGGGAGTAA ACTCGACATA CGTTCTCTAT CACTGATAGG |
| 2761 | GAGTAAACTC GACATCGTTC TCTATCACTG ATAGGGAGTA AACTCGACAT ACGTTCTCTA |
| 2821 | TCACTGATAG GGAGTAAACT CGACGCTAGC ATAACTTCGT ATAGCATACA TTATACGAAG |
| 2881 | TTATTCTAGC GCGGCCGCGT TTAAACGAGC TCACTAGTCT CGAGCCCGGG ATCGACTGCA |
| 2941 | GCCAAGCTTG GCGTAATCAT GGTCATAGCT GTTTCCTGTG TGAAATTGTT ATCCGCTCAC |
| 3001 | AATTCCACAC AACATACGAG CCGGAAGCAT AAAGTGTAAA GCCTGGGGTG CCTAATGAGT |
| 3061 | GAGGTAACTC ACATTAATTG CGTTGCGCTC ACTGCCCGCT TTCCAGTCGG GAAACCTGTC |
| 3121 | GTGCCAGCTG CATTAATGAA TCGGCCAACG CGCGGGGAGA GGCGGTTTGC GTATTGGCGC |
| 3181 | TCTTCCGCTT CCTCGCTCAC TGACTCGCTG CGCTCGGTCG TTCGGCTGCG GCGAGCGGTA |
| 3241 | TCAGCTCACT CAAAGGCGGT AATACGGTTA TCCACAGAAT CAGGGGATAA CGCAGGAAAG |
| 3301 | AACATGTGAG CAAAAGGCCA GCAAAAGGCC AGGAACCGTA AAAAGGCCGC GTTGCTGGCG |
| 3361 | TTTTTCCATA GGCTCCGCCC CCCTGACGAG CATCACAAAA ATCGACGCTC AAGTCAGAGG |
| 3421 | TGGCGAAACC CGACAGGACT ATAAAGATAC CAGGCGTTTC CCCCTGGAAG CTCCCTCGTG |
| 3481 | CGCTCTCCTG TTCCGACCCT GCCGCTTACC GGATACCTGT CCGCCTTTCT CCCTTCGGGA |
| 3541 | AGCGTGGCGC TTTCTCAATG CTCACGCTGT AGGTATCTCA GTTCGGTGTA GGTCGTTCGC |
| 3601 | TCCAAGCTGG GCTGTGTGCA CGAACCCCCC GTTCAGCCCG ACCGCTGCGC CTTATCCGGT |
| 3661 | AACTATCGTC TTGAGTCCAA CCCGGTAAGA CACGACTTAT CGCCACTGGC AGCAGCCACT |
| 3721 | GGTAACAGGA TTAGCAGAGC GAGGTATGTA GGCGGTGCTA CAGAGTTCTT GAAGTGGTGG |
| 3781 | CCTAACTACG GCTACACTAG AAGGACAGTA TTTGGTATCT GCGCTCTGCT GAAGCCAGTT |
| 3841 | ACCTTCGGAA AAAGAGTTGG TAGCTCTTGA TCCGGCAAAC AAACCACCGC TGGTAGCGGT |
| 3901 | GGTTTTTTTG TTTGCAAGCA GCAGATTACG CGCAGAAAAA AAGGATCTCA AGAAGATCCT |
| 3961 | TTGATCTTTT CTACGGGGTC TGACGCTCAG TGGAACGAAA ACTCACGTTA AGGGATTTTG |
| 4021 | GTCATGAGAT TATCAAAAAG GATCTTCACC TAGATCCTTT TAAATTAAAA ATGAAGTTTT |
| 4081 | AAATCAATCT AAAGTATATA TGAGTAAACT TGGTCTGACA GTTACCAATG CTTAATCAGT |
| 4141 | GAGGCACCTA TCTCAGCGAT CTGTCTATTT CGTTCATCCA TAGTTGCCTG ACTCCCCGTC |
| 4201 | GTGTAGATAA CTACGATACG GGAGGGCTTA CCATCTGGCC CCAGTGCTGC AATGATACCG |
| 4261 | CGAGACCCAC GCTCACCGGC TCCAGATTTA TCAGCAATAA ACCAGCCAGC CGGAAGGGCC |
| 4321 | GAGCGCAGAA GTGGTCCTGC AACTTTATCC GCCTCCATCC AGTCTATTAA TTGTTGCCGG |
| 4381 | GAAGCTAGAG TAAGTAGTTC GCCAGTTAAT AGTTTGCGCA ACGTTGTTGC CATTGCTACA |
| 4441 | GGCATCGTGG TGTCACGCTC GTCGTTTGGT ATGGCTTCAT TCAGCTCCGG TTCCCAACGA |
| 4501 | TCAAGGCGAG TTACATGATC CCCCATGTTG TGCAAAAAAG CGGTTAGCTC CTTCGGTCCT |
| 4561 | CCGATCGTTG TCAGAAGTAA GTTGGCCGCA GTGTTATCAC TCATGGTTAT GGCAGCACTG |
| 4621 | CATAATTCTC TTACTGTCAT GCCATCCGTA AGATGCTTTT CTGTGACTGG TGAGTACTCA |
| 4681 | ACCAAGTCAT TCTGAGAATA GTGTATGCGG CGACCGAGTT GCTCTTGCCC GGCGTCAATA |
| 4741 | CGGGATAATA CCGCGCCACA TAGCAGAACT TTAAAAGTGC TCATCATTGG AAAACGTTCT |
| 4801 | TCGGGGCGAA AACTCTCAAG GATCTTACCG CTGTTGAGAT CCAGTTCGAT GTAACCCACT |
| 4861 | CGTGCACCCA ACTGATCTTC AGCATCTTTT ACTTTCACCA GCGTTTCTGG GTGAGCAAAA |
| 4921 | ACAGGAAGGC AAAATGCCGC AAAAAAGGGA ATAAGGGCGA CACGGAAATG TTGAATACTC |
| 4981 | ATACTCTTCC TTTTTCAATA TTATTGAAGC ATTTATCAGG GTTATTGTCT CATGAGCGGA |
| 5041 | TACATATTTG AATGTATTTA GAAAAATAAA CAAATAGGGG TTCCGCGCAC ATTTCCCCGA |
| 5101 | AAAGTGCCAC CTGACGTCTA AGAAACCATT ATTATCATGA CATTAACCTA TAAAAATAGG |
| 5161 | CGTATCACGA GGCCCTTTCG TCTTCAAGAA// |
| SEQUENCE NO. 2 | |
| HSC-CRE-ERt | |
| LOCUS Untitled 13033 bp DNA linear SYN 03-JAN.-2008 | |
| DEFINITION. | |
| ACCESSION. | |
| KEYWORDS. | |
| FEATURES Location/Qualifiers | |
| BASE COUNT 3087 a 31210 c 3427 g 3399 t | |
| ORIGIN | |
| 1 | ACGACTGGAG AGATGGCTCA CTGGTTAAGA GCACTGACTA TTCTTCCAGA GGTCCTGAGT |
| 61 | TCAATTCCCA ACAACCACAT GGTGGCTCAG AACCATCTGT AATGGGATCT GATGCCCTCT |
| 121 | CCTAGTGTGT CTGAAGGCAG CCACAGTGTG TGTGTGTGTG TGTGTGTGTG TGTATACATA |
| 181 | TACATATATA TGTATATATA TAATTTTTGC ATATTAAATC TATAAAAAAA AAACCCAGTG |
| 241 | AGATCCGAGT TCTGTGTATT GAGAATACCA AGGTGTATGG TGTGTGTGTG TGGGGGGGAA |
| 301 | GAGGACACTT CATTGGAATA ATTCAAGGAA GAGCTTTCTT TATATTTTCT CCATCAGGAG |
| 361 | GGGAGCCCAG ATTCTAGTGA CTTCTGGAGC ACTTTCCCAA GTCTTAAGAG TCCAGCTGAG |
| 421 | CAGAATGGGG TGGAGTGTGA AGGGTAGTAG GACCAGAATC CAGGATTAGC TTCAGTCCTT |
| 481 | GACTCCCTTT CTTATGATAG GGTAGCTACT TGCAGAATAC AACGGTGGGT TTGCTTAGTG |
| 541 | TAGGCTGCTT TCCTCTTGGC CGGGAATATT TCTGACATCC TTGGTTGAAT AGAGCAGAGT |
| 601 | TCTTGCAGCT TCCACACCCT ACTTCACCAC CATAGTCTTT CTGGGTGTAT ATTTGCAGCG |
| 661 | CATGTGTGTA GCAGTAGATC GGGAGAGGGT TCCTATAGCA CTGGACAGAT TCCCCGCCAA |
| 721 | AACCAAAAGG GGGGCGGGAA GGACACGCTT GCTCGGGGGA TTAGTTCCCT CCCCTTCCCC |
| 781 | TGTGGCCTAA GAAGGAGGGA CTGGGTGATC TTTCTCTTCT CTGTGCATTT CCTTCCTCCT |
| 841 | TTTTCCCGTC GATTTTTGTC TCTCTGCCTG TATTCCTTTT CTCCCAAGGT TTCTGCCATC |
| 901 | TTTCTCCAGC ACAATTCCTA CCCTTGGACA CTGTGCCTTC CGGGCTTGTC CCACCCTTTT |
| 961 | CTTCCAATCT AGAGACACCC CCACATTGCT CCAGCTCCAG GCCTGTGGGC CTTCACGCCA |
| 1021 | GCAGGGTTGG GGTGTGCGTC CACGTGGTGC TGAGTTTGTC CTGTCCGCTT TTCAGGTTTC |
| 1081 | AGTGCGTGAT CTCCTCTCTG CCCCTTACCC TGTTACAGGA TGACGGAGCG GCCGCCGAGC |
| 1141 | GAGGCGGCAC GCAGTGACCC GCAACTAGAG GGACAGGACG CGGCCGAGGC CCGCATGGCC |
| 1201 | CCCCCGCACC TAGTCCTGCT CAACGGCGTC GCCAAGGAGA CGAGCCGCGC AGCCCCGGCT |
| 1261 | GAGCCCCCCG TCATCGAGCT AGGAGCGCGC AGCGGCGCGG GGGGCGGCCC TGCCAGTGGG |
| 1321 | GGCGGTGCCG CGAGGGACTT AAAGGGCCGC GACGCAGTAG CAGCCGAAGC TCGCCTTCGG |
| 1381 | GTGCCCACCA CCGAGCTGTG CAGACCTCCC GGACCCGCCC CGGCGCCCGC GCCCGCTTCG |
| 1441 | GTTCCTGCAG AGCTGCCTGG AGACGGCCGC ATGGTGCAGC TGAGCCCGCC CGCGCTGGCA |
| 1501 | GCCCCTGCCG GCCCCGGCCG AGCGCTGCTC TATAGCCTTA GCCAGCCGCT CGCCTCACTA |
| 1561 | GGCAGGTGAG CATCCCGGTC CCCTGCGGCG TTCTGGGTGC AGGCGAGGGT CGAGAGGAGG |
| 1621 | GGGTGGTGGC TTAAGATTCC AAGAGGAACG AGCCCAGAGA CCAGAGTCTC TCCCGCAACC |
| 1681 | CTCCCGCTAG TGGGAAAGGG GTCCCCTGTG AGACAGACTG TCAGGAAGGA CCGGTGGTCA |
| 1741 | GGGGACGACA GTTGTGTAGA AACCGGGGGT GGTCGCCTGC ACTGTTGAGG GTGCGGGTCT |
| 1801 | GTGGGTGAGT GTAAAAAGCT GCAGAGGTTG CTGACTACTG TTGAGTAGGC GGGATTCTTT |
| 1861 | AATATGAGTT CTGGGCCAGT GTCTGAATGC CCCTCTGCAG CAGAGGTGAG GTTCGCCACA |
| 1921 | AAGGGTGAAC TCTTCAGGAA GCTGCCGCGG TGGGTGGACA GGCTGGAGAG AAAGATCTAA |
| 1981 | GGCCGTTGCT GAGGGCAGCT CTTCTCAGCC TCTGCTAGGA TGCAGTGAGC GACACTGTCA |
| 2041 | TCCGCTCCTA ATCCTTCTGT CCCTTACCTG CGTGGTTGGT CTCCTTGCTG GGCCCTGTGG |
| 2101 | TGAGGGAAGC TGAATGGCCA GCAGAGTGTA GGACAGGCGG TAGGAAAGAA TTATAGGACA |
| 2161 | ACACGATGGT AGAGCAGTAG GGAGCGCTGT CAAGGGTTGG TGAGTGGGAG GTGGGGGGTG |
| 2221 | GTGCCGATCT GTGATCAGAG AGTGATGGTC GGTGAGGTCT GAGGGGACAA TGTGAGACCC |
| 2281 | TTTGTGGTGT GGGAGTTCTC TACTAGCACT TCCATCCCTC ACGTGTTGTC CTGTGTAGGT |
| 2341 | ACTTGTCTCT GAGCAAAGGT CTACCAGGAT TGAAGGAGAT TTTGTGTGTG TGTGTGTGTG |
| 2401 | TGTGTGTGTG TGTGTGTGTG TGTGTGTGTA CTTCAGCACA GGAATACGCC GCCTTGCCCC |
| 2461 | TCCCATTTAT GTATTGTTCC ATATATTCAC CCTCTTCGCT TCTGTGAATG CATGCATACT |
| 2521 | CAATTCAATC TGCATTTTAA GTGTGCAGGA GCAGGGGGTG CCTTAGCAGG AGGGGACTGA |
| 2581 | AGACACACAG GGAGAATCCA TCTAAGGAGT CTTTTTGTCT TTAACCTCAT TGTGATCTAC |
| 2641 | CTTCTCTTTC CATAGTGGGT TCTTTGGGGA ACCGGATGCC TTCCCCATGT TCACCAACAA |
| 2701 | CAACCGGGTG AAGAGGAGGC CCTCCCCATA AATTCCACCA TGTCCAATTT ACTGACCGTA |
| 2761 | CACCAAAATT TGCCTGCATT ACCGGTCGAT GCAACGAGTG ATGAGGTTCG CAAGAACCTG |
| 2821 | ATGGACATGT TCAGGGATCG CCAGGCGTTT TCTGAGCATA CCTGGAAAAT GCTTCTGTCC |
| 2881 | GTTTGCCGGT CGTGGGCGGC ATGGTGCAAG TTGAATAACC GGAAATGGTT TCCCGCAGAA |
| 2941 | CCTGAAGATG TTCGCGATTA TCTTCTATAT CTTCAGGCGC GCGGTCTGGC AGTAAAAACT |
| 3001 | ATCCAGCAAC ATTTGGGCCA GCTAAACATG CTTCATCGTC GGTCCGGGCT GCCACGACCA |
| 3061 | AGTGACAGCA ATGCTGTTTC ACTGGTTATG CGGCGGATCC GAAAAGAAAA CGTTGATGCC |
| 3121 | GGTGAACGTG CAAAACAGGC TCTAGCGTTC GAACGCACTG ATTTCGACCA GGTTCGTTCA |
| 3181 | CTCATGGAAA ATAGCGATCG CTGCCAGGAT ATACGTAATC TGGCATTTCT GGGGATTGCT |
| 3241 | TATAACACCC TGTTACGTAT AGCCGAAATT GCCAGGATCA GGGTTAAAGA TATCTCACGT |
| 3301 | ACTGACGGTG GGAGAATGTT AATCCATATT GGCAGAACGA AAACGCTGGT TAGCACCGCA |
| 3361 | GGTGTAGAGA AGGCACTTAG CCTGGGGGTA ACTAAACTGG TCGAGCGATG GATTTCCGTC |
| 3421 | TCTGGTGTAG CTGATGATCC GAATAACTAC CTGTTTTGCC GGGTCAGAAA AAATGGTGTT |
| 3481 | GCCGCGCCAT CTGCCACCAG CCAGCTATCA ACTCGCGCCC TGGAAGGGAT TTTTGAAGCA |
| 3541 | ACTCATCGAT TGATTTACGG CGCTAAGGAT GACTCTGGTC AGAGATACCT GGCCTGGTCT |
| 3601 | GGACACAGTG CCCGTGTCGG AGCCGCGCGA GATATGGCCC GCGCTGGAGT TTCAATACCG |
| 3661 | GAGATCATGC AAGCTGGTGG CTGGACCAAT GTAAATATTG TCATGAACTA TATCCGTAAC |
| 3721 | CTGGATAGTG AAACAGGGGC AATGGTGCGC CTGCTGGAAG ATGGCGATCT CGAGCCATCT |
| 3781 | GCTGGAGACA TGAGAGCTGC CAACCTTTGG CCAAGCCCGC TCATGATCAA ACGCTCTAAG |
| 3841 | AAGAACAGCC TGGCCTTGTC CCTGACGGCC GACCAGATGG TCAGTGCCTT GTTGGATGCT |
| 3901 | GAGCCCCCCA TACTCTATTC CGAGTATGAT CCTACCAGAC CCTTCAGTGA AGCTTCGATG |
| 3961 | ATGGGCTTAC TGACCAACCT GGCAGACAGG GAGCTGGTTC ACATGATCAA CTGGGCGAAG |
| 4021 | AGGGTGCCAG GCTTTGTGGA TTTGACCCTC CATGATCAGG TCCACCTTCT AGAATGTGCC |
| 4081 | TGGCTAGAGA TCCTGATGAT TGGTCTCGTC TGGCGCTCCA TGGAGCACCC AGGGAAGCTA |
| 4141 | CTGTTTGCTC CTAACTTGCT CTTGGACAGG AACCAGGGAA AATGTGTAGA GGGCATGGTG |
| 4201 | GAGATCTTCG ACATGCTGCT GGCTACATCA TCTCGGTTCC GCATGATGAA TCTGCAGGGA |
| 4261 | GAGGAGTTTG TGTGCCTCAA ATCTATTATT TTGCTTAATT CTGGAGTGTA CACATTTCTG |
| 4321 | TCCAGCACCC TGAAGTCTCT GGAAGAGAAG GACCATATCC ACCGAGTCCT GGACAAGATC |
| 4381 | ACAGACACTT TGATCCACCT GATGGCCAAG GCAGGCCTGA CCCTGCAGCA GCAGCACCAG |
| 4441 | CGGCTGGCCC AGCTCCTCCT CATCCTCTCC CACATCAGGC ACATGAGTAA CAAAGGCATG |
| 4501 | GAGCATCTGT ACAGCATGAA GTGCAAGAAC GTGGTGCCCC TCTATGACCT GCTGCTGGAG |
| 4561 | ATGCTGGACG CCCACCGCCT ACATGCGCCC ACTAGCCGTG GAGGGGCATC CGTGGAGGAG |
| 4621 | ACGGACCAAA GCCACTTGGC CACTGCGGGC TCTACTTCAT CGCATTCCTT GCAAAAGTAT |
| 4681 | TACATCACGG GGGAGGCAGA GGGTTTCCCT GCCACAGTCT GAGAGCTCCC TGGCGGAATT |
| 4741 | CGGATCTTAT TAAAGCAGAA CTTGTTTATT GCAGCTTATA ATGGTTACAA ATAAAGCAAT |
| 4801 | AGCATCACAA ATTTCACAAA TAAAGCATTT TTTTCACTGC ATTCTAGTTG TGGTTTGTCC |
| 4861 | AAACTCATCA ATGTATCTTA TCATGTCTGG TCGAGATCTA AGGAAGACCC TGAATTCTGT |
| 4921 | TCTCATACTC CATACCCCAT ATCTTTCTTC CTCTGTGTCT TCCTTGCCCT TAAAGAAATT |
| 4981 | GCAGCATTCC AAGAACAATA TCTGTACAAA GGGGGAAATG TAAGCATGAG AAAACATTAA |
| 5041 | AAAAAAAAAA CAGTGATGAA CATAACCACA GAGAGAATCC CACCCTTCAA GAATAATTCA |
| 5101 | TGTTTATTTG TGGTGGCAAA TAACAAAATG GTACAACCTT TATCCTTTTC CAGAAACAAA |
| 5161 | AACCAAGGGC ACAGCAACTA GAGTGAGCTG ACAGCTATTT TGGCCTTTTT GGTGGGTCTA |
| 5221 | GCCGTACTTG GGATCCCAGT GGTACATGAC CCTCTGCCGA AGGCTTGCCT CAGTCTGTGT |
| 5281 | ACATAGCACG CCATGTCTGT GGGCAAGCCC AGCACTTTGC GTCAGTGTCG TACTGTATGT |
| 5341 | AATGAACTGT GTTGGTCTCT GTGTTTTTTT TTTCTGAAGA AGAGGAGTAA CTACTCCGGG |
| 5401 | TACCTTGATA TTTGTACAGC CTATAGGCCA ACACTGCGGG CGTGTGACTC TTTATTGAAA |
| 5461 | AACAAAAACA AAAAAATACC AGTGTGGTGA TGATAGTGTG TGTATATATA TATAAGGTTA |
| 5521 | TATGGGGAAG ATTTCTAAAT AAAAGTTTTA CAAAGGGGCC TGGACTTTGT ACTTGGACTT |
| 5581 | TGCCCCCTAG AGTCTGAGAA TGGGAACATC AAGGGGAAAG GCTGACAGCT TTTAGGAAGT |
| 5641 | AGGATCTAGC TTCCAGTCTC AGCCTGTCGG GGAGGAAGGA GGCTACCCTA TGGGGGGGTT |
| 5701 | TCCTTTTCCC CCCTTCTGCA AGGCTCCAAG GGCTTCAGTA TCCTGTCCTT GTGTTTGCAG |
| 5761 | CCCTAGACAG CCTAGACCTC TCTGTGTAGG GTCAGCTTTC TCCTTGTTAG ATCACTTTCC |
| 5821 | CAAGTTGGGA CCATTGCTCC CAGTGAGAGC TTAGGACAGA AAAATGTAGC TGTTATCCAC |
| 5881 | CATTGGTGTC CATAGATTTC CTGATGACTC AGTGGGGGTT GCATCTTTTA CACTTGACTT |
| 5941 | TTTTTTTAAA GGTTAAAAAA TATTTTATGT ATAGAGATGT TTTATGTGTA TAGGTACAGT |
| 6001 | GCCCACAGAT GCCAGAAAAG GGAGTCGGAT TCCCTGGAAC TGGAGTTGCA AACCGTTGTG |
| 6061 | AGTTGCCCTG TAGGTGCTGG AGTTTCATGA ATAGAATTTG GGAAAGAGAC TGGGTCTTGG |
| 6121 | GGAGGCCATT ATGCATGGAC GTTTGGTCTC CTGGGAGTTT GTAAGCTGGG CATCTTCTGT |
| 6181 | CTTCTCATTT AACAAGCATT TGCTGAGCTC CTGCTCTGGG CAGACACTGT TCTGTTGGGG |
| 6241 | AGGGTTCAGC ATTGAATGAA ACAAGCATGG ATGCTCTCCA CTGCACCTTA CATTTTAGCA |
| 6301 | GGGGGATGTT GAATGCAGAA ACACATACAA GTAGAGTTAA ATAGTTAGAA AGCAAATTAG |
| 6361 | TATTAACCCA CAGTGAGTTT TATTCAGGCC AGCCTGGGCT ACAGTCTCAA AAACCAAAGC |
| 6421 | CAAGAAAGGT GGTAAGGAAC AAAAGTGGGC AGATCAACAG GGATAGTTCA GGAAGGCCC |
| 6481 | TAGGGTGCCA TCTTTTTCAT TCAGGATCAG ATGATTCCTG GTGTCAGAGA CAGTTTTGTC |
| 6541 | CCAGGGACAG GTTGGGTCTT TCTATCTACA TGCCCTGAGA TGGCTTTTTT CTTTCTTCTT |
| 6601 | CTCTGGACCT CAGTACTCAA CCCCAAATCT ACAGACATGG ACTAGCTCAG ATTCAACAAT |
| 6661 | TGGGAGGGAA TTCAATAGTC TCACCGTTAA TTCCCAGCTG GCCTGTCTCT AGTCTCAGCT |
| 6721 | GTGTTTTGTC CTCTTAGCTT CTATCCATCT ACAGGGAGAG GGTAGGATTC AGCCTGAGTG |
| 6781 | TCAATATCTG ATCCAGCTAC TGGGAAGCTC CTCAGATATG CCTCTCTTTG GCCTAGGACA |
| 6841 | AGGATGGTAG GATTTGGCCT TGGGGAGGGG AGAAAAATGG ATATTTAGGC TTATAGACCT |
| 6901 | GAGGAACTAT CATGATAGGA GAGAAAGAAA GAGGACAGAG AAGGAAGAAT GTGTTTGGG |
| 6961 | GTGGAGGAAG TGGCCAGTAT GCTCAGTACA ACTGAGGGGC CATGCACGGA AAGGCTGAGT |
| 7021 | TAACTGGTTT GAGGCAGCTG GTGACTGGAA AGAGCTGCAG AGAGGAGTGA ATAGAGGTAG |
| 7081 | TGACCTGAGG ACTCAGAGAT GTCACTTCCC ATCTTGTAAG ATTTTCCTCA GGAGAAATGA |
| 7141 | AGCTTTCCAT GTAATGGTGA CAAAGAGAGC CCGAGGATTC TGATCACTCC CGGAGTTCAT |
| 7201 | CGATGGGGCA GAGACCCAGA GAGAAAATGT CTTCTCAAGC CTTGTATCTC AGAGTGGTGT |
| 7261 | GTAGGCAGGC CCATTCTCCC TGTCCCAAGA AAATGTTGTC TCTGAAGCCC AGAATCCCTG |
| 7321 | ACTCCACAAG GGAAGAAAAG TGCCCTGAGG CCTGGCCTGA GGTGTTTTGC TGATCTGTTC |
| 7381 | CCCTTTATTT CTTACCACTC CATTTGTGTG TGTGTGTGTG TGTGTGTGTG TGTTTGCTTA |
| 7441 | TTTGTTTTTC TGAAACAGGG TCTCATGTGG CCTCAAACCC ACTAAGTTGC TGAGGCTGAC |
| 7501 | TTTGAACTTC CGATCTTCCT GCCTCTGTCT CCAGAGTGCT GGGATTACAG GTGTGCACTA |
| 7561 | GAATACCAGG TTTATTCAGT GACAGGAGCT AAATCCAGAG CTTTGTGCAT ATTAGGCAAG |
| 7621 | CACTCTACAA CCAGACTGCA TCCCCACCCC ACGCCTCACT CTTTTGTGCC TACCGTACTA |
| 7681 | GCTTTCTTCC TTTTTGTTTT AGACTGTTTT ATTGGTTTTT GACTCCCAGA TGTTGAATTT |
| 7741 | TGGTTTATTT TTCACATAAC AGCCCATCTT CCTCTTTGCC CACTCTCATT TGGTTGAATT |
| 7801 | GTCCCTGAAG TCCAGGAAGT TTTCCTGACT CCATGGGACT GGGTGCCTCC TTTGCATCCC |
| 7861 | CATGGGACCC CAGGTATGCT GGCCCTTCCT GCCCTAACAT TTGCTTATTT AGTTGCTTCT |
| 7921 | TCACTGAAAC ACAAACCCCT CAGAGCTGAA ACCAAGTCTG ATTAAGCCCT CTGCACCAGC |
| 7981 | ACCTTAGGGT ACAGACACTC GGTTCTTTCC CCACTGGCCA TGAACAGCCC TTCTCCTCCC |
| 8041 | ACTGGCTCTC TATTTTCTCT CTGGGCCTGG CGTCTGACCT GGCATCTGGC AAGGACCTGA |
| 8101 | AAGGCTGGTA TAGAGTGGTG AAGACCAGGC ATGGAGGCTA TGGATCCAGT CAGCTGTCTG |
| 8161 | GCCTCCTCAC GCCGGTCCCT ACCTGCTTCC TTTTTAATAA AATAAGTGTG TGTTCCTCAG |
| 8221 | AAGCTGTCAC TGTGTCATTA GCTTCCTCGC ACCCCCTACC CGGACACACC CCCCTGCCCA |
| 8281 | TGTAAACCTG TTACCTATTC ACAGAGCTTA ATTGTCATGA ATCTAAGTAA AGGGTTACCC |
| 8341 | AGGGGAGGTG ACACAAAGCC CTGAGTTGGA AGGGGCTTGA GCAAGGTGAA GTAGGTGTGA |
| 8401 | ATTCAGGGCG ACACCCAAGG TTAGAGATCC AGACCACATA GGAAGGTCAG GAAATAGAAG |
| 8461 | AGGAGGCCAG TAGACAGCTA GAGTTCATAG AGAAAATGGC TTTACTTTCC TTATGGGCAA |
| 8521 | GAGGGCTACA CAAATTTAGG CCCAGGACAG GTGGTGGTAG TGAAGAGCTT GCTGGCTGGA |
| 8581 | GGACTGGCTC TGTGGATGAC CATGGGGACA GTGAGGAAGG ACAGTTGGTG TGGAACAGTT |
| 8641 | GGTGAAGGGA GTAACTGGGG CCTGGGTGGA AGTGAGAAGA AAAGAGCAGC CAGGCTCTGG |
| 8701 | AGGAGCTTGG CCTGGTCAGA ATCACTTGGG GCTTAAGGGC TTAAGTATTG CTACTGGGTG |
| 8761 | TGCTGGCTTG TGACTTTGAG TGAGTCACTA TCATTCTGAG GTTTGGTTTC TTTATCTGTT |
| 8821 | AAACAGAGAT GTTAACAGTC ATCTTCCAGG ACTGTCATGG GACTTCAGCA TAATATATGC |
| 8881 | AAAGTATCTG TGTTTCATTA AAAAATGATT CTATAGAAAG AGCTACGGAA ATATCTATAA |
| 8941 | GAAAGCATTC TTTTTCCAAG AAACAGGACC AGGAGGGATG GGACTGTCCT AACAGAAGAG |
| 9001 | ACGAGGGAAG GACATGAGTG TGAGGGAATA TTAATCCCTC ACTCAACAGC AGGACTTTTG |
| 9061 | TGTGCCTGTC TTATGTCAGG AAAGGAGGGG TAGCCAGTCT TGACCACCCA TTTTGACTTC |
| 9121 | AGAGGCTGGA GAGCAGAGTG GAAGCTGGGA ATAGGAAGGA ATCCTAGAGG CAAGTGCTAT |
| 9181 | GGGAGGAGCT TAGTGGTGTG GTGTGGGCAG CCTAGCTCTG ACAGTAAAGT CCCTGAGCAA |
| 9241 | GTTGTGCTGA ACTGAACTGT CCTGAGGGGC AAGGTTGGGA GGTATCTGGG AGATTTCACA |
| 9301 | TTCTGTCTTG AGCATTACCT AGTTTTCAGT GGTGGAGCGG GCTGGTCCAG GAATGCTGGC |
| 9361 | TTCCTCCTGG GCCCCATACT CTTGCCAAGG CTACCTGGGG TGAGGCAATG CTCCCCCACC |
| 9421 | TCACTTTGCC TTCCAGCTCC TACTTAAGCT CTCCCCACTG GTTTGCTCTG AGGCCTGCCC |
| 9481 | CTCCCCAGCT CCTGGGCTTT CTCTCCACAC AATAACAGGA TGTGATCTTC GAAGAGAGGA |
| 9541 | AGTGGGGGAG GACTGCTGTG CCGATAGCAG GGAAGGAGGG GGGCTTCTGA CTCTCCCCTC |
| 9601 | TCCAGCCCTC CTTTGCTCTG TAGGCCAGCC CCTGCAGCTC CTTGATCCCC CTAAGCCCTA |
| 9661 | CCTCAAGCTT CTATCTGAGA CAAGTAGGGA TGAAGGGTCT TTAGGCCCAT GTAGGACTGC |
| 9721 | TTGCCTATGG AGAGACATGC CTTGGCCACA CCGTCTTCAG GATCTACCTT CTGGAGAGAC |
| 9781 | TTGCTGGCCT AGCTTTAGAT GCTGGGTTGT TTTCTGCCCG GAGCTGCTGG AGTCTAAGGG |
| 9841 | TGGGCAGGTG GGTCATTCTG TAGGGCTCCA TCTGTCCAGT GCACTCCCAA GTCCACACGA |
| 9901 | GCATGATTCA GTGCAGGGAG TGCGTGATAG CATCAATCTA AAGGTCTATG TCAAATGCTG |
| 9961 | GTTTGGCTTG CACAGTGTGT GTCAGGCTGC AAAAATGGAC AGTGAAATCC AGAAAGACAA |
| 10021 | GGAGCATGAG GAAGGAGCAA GGCTAGGCTG GAACCCAGCA CTAGGTCATT GGGTTACCGC |
| 10081 | CTCTTCGAGC CAGGGATGTT CTTAGAACTT CCAAAGTTGA TGGGAAAGTT TTAGATCGAG |
| 10141 | TCGACCGATG CCCTTGAGAG CCTTCAACCC AGTCAGCTCC TTCCGGTGGG CGCGGGGCAT |
| 10201 | GACTATCGTC GCCGCACTTA TGACTGTCTT CTTTATCATG CAACTCGTAG GACAGGTGCC |
| 10261 | GGCAGCGCTC TTCCGCTTCC TCGCTCACTG ACTCGCTGCG CTCGGTCGTT CGGCTGCGGC |
| 10321 | GAGCGGTATC AGCTCACTCA AAGGCGGTAA TACGGTTATC CACAGAATCA GGGGATAACG |
| 10381 | CAGGAAAGAA CATGTGAGCA AAAGGCCAGC AAAAGGCCAG GAACCGTAAA AGGCCGCGT |
| 10441 | TGCTGGCGTT TTTCCATAGG CTCCGCCCCC CTGACGAGCA TCACAAAAAT CGACGCTCAA |
| 10501 | GTCAGAGGTG GCGAAACCCG ACAGGACTAT AAAGATACCA GGCGTTTCCC CCTGGAAGCT |
| 10561 | CCCTCGTGCG CTCTCCTGTT CCGACCCTGC CGCTTACCGG ATACCTGTCC GCCTTTCTCC |
| 10621 | CTTCGGGAAG CGTGGCGCTT TCTCATAGCT CACGCTGTAG GTATCTCAGT TCGGTGTAGG |
| 10681 | TCGTTCGCTC CAAGCTGGGC TGTGTGCACG AACCCCCCGT TCAGCCCGAC CGCTGCGCCT |
| 10741 | TATCCGGTAA CTATCGTCTT GAGTCCAACC CGGTAAGACA CGACTTATCG CCACTGGCAG |
| 10801 | CAGCCACTGG TAACAGGATT AGCAGAGCGA GGTATGTAGG CGGTGCTACA GAGTTCTTGA |
| 10861 | AGTGGTGGCC TAACTACGGC TACACTAGAA GAACAGTATT TGGTATCTGC GCTCTGCTGA |
| 10921 | AGCCAGTTAC CTTCGGAAAA AGAGTTGGTA GCTCTTGATC CGGCAAACAA ACCACCGCTG |
| 10981 | GTAGCGGTGG TTTTTTTGTT TGCAAGCAGC AGATTACGCG CAGAAAAAAA GGATCTCAAG |
| 11041 | AAGATCCTTT GATCTTTTCT ACGGGGTCTG ACGCTCAGTG GAACGAAAAC TCACGTTAAG |
| 11101 | GGATTTTGGT CATGAGATTA TCAAAAAGGA TCTTCACCTA GATCCTTTTA AATTAAAAAT |
| 11161 | GAAGTTTTAA ATCAATCTAA AGTATATATG AGTAAACTTG GTCTGACAGT TACCAATGCT |
| 11221 | TAATCAGTGA GGCACCTATC TCAGCGATCT GTCTATTTCG TTCATCCATA GTTGCCTGAC |
| 11281 | TCCCCGTCGT GTAGATAACT ACGATACGGG AGGGCTTACC ATCTGGCCCC AGTGCTGCAA |
| 11341 | TGATACCGCG AGACCCACGC TCACCGGCTC CAGATTTATC AGCAATAAAC CAGCCAGCCG |
| 11401 | GAAGGGCCGA GCGCAGAAGT GGTCCTGCAA CTTTATCCGC CTCCATCCAG TCTATTAATT |
| 11461 | GTTGCCGGGA AGCTAGAGTA AGTAGTTCGC CAGTTAATAG TTTGCGCAAC GTTGTTGCCA |
| 11521 | TTGCTACAGG CATCGTGGTG TCACGCTCGT CGTTTGGTAT GGCTTCATTC AGCTCCGGTT |
| 11581 | CCCAACGATC AAGGCGAGTT ACATGATCCC CCATGTTGTG CAAAAAAGCG GTTAGCTCCT |
| 11641 | TCGGTCCTCC GATCGTTGTC AGAAGTAAGT TGGCCGCAGT GTTATCACTC ATGGTTATGG |
| 11701 | CAGCACTGCA TAATTCTCTT ACTGTCATGC CATCCGTAAG ATGCTTTTCT GTGACTGGTG |
| 11761 | AGTACTCAAC CAAGTCATTC TGAGAATAGT GTATGCGGCG ACCGAGTTGC TCTTGCCCGG |
| 11821 | CGTCAATACG GGATAATACC GCGCCACATA GCAGAACTTT AAAAGTGCTC ATCATTGGAA |
| 11881 | AACGTTCTTC GGGGCGAAAA CTCTCAAGGA TCTTACCGCT GTTGAGATCC AGTTCGATGT |
| 11941 | AACCCACTCG TGCACCCAAC TGATCTTCAG CATCTTTTAC TTTCACCAGC GTTTCTGGGT |
| 12001 | GAGCAAAAAC AGGAAGGCAA AATGCCGCAA AAAAGGGAAT AAGGGCGACA GGAAATGTT |
| 12061 | GAATACTCAT ACTCTTCCTT TTTCAATATT ATTGAAGCAT TTATCAGGGT TATTGTCTCA |
| 12121 | TGAGCGGATA CATATTTGAA TGTATTTAGA AAAATAAACA AATAGGGGTT CCGCGCACAT |
| 12181 | TTCCCCGAAA AGTGCCACCT GACGCGCCCT GTAGCGGCGC ATTAAGCGCG GCGGGTGTGG |
| 12241 | TGGTTACGCG CAGCGTGACC GCTACACTTG CCAGCGCCCT AGCGCCCGCT CCTTTCGCTT |
| 12301 | TCTTCCCTTC CTTTCTCGCC ACGTTCGCCG GCTTTCCCCG TCAAGCTCTA AATCGGGGGC |
| 12361 | TCCCTTTAGG GTTCCGATTT AGTGCTTTAC GGCACCTCGA CCCCAAAAAA CTTGATTAGG |
| 12421 | GTGATGGTTC ACGTAGTGGG CCATCGCCCT GATAGACGGT TTTTCGCCCT TTGACGTTGG |
| 12481 | AGTCCACGTT CTTTAATAGT GGACTCTTGT TCCAAACTGG AACAACACTC AACCCTATCT |
| 12541 | CGGTCTATTC TTTTGATTTA TAAGGGATTT TGCCGATTTC GGCCTATTGG TTAAAAAATG |
| 12601 | AGCTGATTTA ACAAAAATTT AACGCGAATT TTAACAAAAT ATTAACGCTT ACAATTTGCC |
| 12661 | ATTCGCCATT CAGGCTGCGC AACTGTTGGG AAGGGCGATC GGTGCGGGCC TCTTCGCTAT |
| 12721 | TACGCCAGCC CAAGCTACCA TGATAAGTAA GTAATATTAA GGTACGTGGA GGTTTTACTT |
| 12781 | GCTTTAAAAA CCTCCCACAC CTCCCCCTGA ACCTGAAACA TAAAATGAAT GCAATTGTTG |
| 12841 | TTGTTAACTT GTTTATTGCA GCTTATAATG GTTACAAATA AAGCAATAGC ATCACAAATT |
| 12901 | TCACAAATAA AGCATTTTTT TCACTGCATT CTAGTTGTGG TTTGTCCAAA CTCATCAATG |
| 12961 | TATCTTATGG TACTGTAACT GAGCTAACAT AACCCGGGAG GTACCGAGCT CTTACGCGTG |
| 13021 | CTAGCTCGAG ATC |
| // | |
| SEQUENCE NO. 3 | |
| Endothelial-CRE-ERt | |
| LOCUS Endo-pGL2-Promoter 12191 bp DNA circular SYN 12-DEC.-2007 | |
| DEFINITION. | |
| ACCESSION. | |
| KEYWORDS. | |
| FEATURES Location/Qualifiers | |
| BASE COUNT 3021 a 3013 c 2707 g 3451 t | |
| ORIGIN | |
| 1 | CCCGGGAGGT ACCCTGGGCT ACACAGAGAT AGATGTCTTT TGCCACAGCT TCTCCTGGCA |
| 61 | ACCCAAAGCT ACCTGGCAGA GTCCAGTCTG CCTAACACCT ATGAATCTAT GAGATACCTT |
| 121 | AAAAAGCATA TCCTTCTTCT ATACATCTTT CCACTTCCTC CTCTTCTCCA CCCTATTCAT |
| 181 | CAGACAACTG TCTCAGTCAG TGGGGAACAT GAAGAGGGGA TATGGATGCT TGCTTTCACA |
| 241 | GGTGCCTCTG CATAAAGGGA GTTCTCAGTG AGCTGGAGCA GAGGCTATGG AGGAAGGAAG |
| 301 | CAGAGATGAC AGATTAAAGA CAGATGCAGA GACAAGGCCT TTATACAGGA AAGAGGAGCA |
| 361 | GATTCAGAGT TTGCCTGAGC CTAAGATAGA GACCGGAGAA ATGAAAGGCA GAGTGAGCAA |
| 421 | GATAAGAGAT GAAAGGAATA GACCCCGGGG TTCCTCCACT GCATCCTCAT TACAGATAGG |
| 481 | AAAACTGAAG GTCAGAGGAA GTGGTGGTTC TACTTCCTAC GGATGTATCC ATCACTCTTG |
| 541 | TAAATACACT GGGTCAGGTC CTCCCTTCTC CAGCACTTTC CTCTTGCCCT TGTGCACTAG |
| 601 | GACTGAGTAA CACAAGTGAC ACCCAGTGGG AGGCTCTTGG ACAAGTCAAC CAGGAAGAGG |
| 661 | GAGAGAGGAG ACAGTGTAGA CAAAATAGAT TGACAGGGAA GTTTTTCTGG ACTAGGTAAG |
| 721 | CTTGAAGAAG GGCACAGAGG GTGTAAACAA CTGTATTAAG GTATATGGTT TATGTGCAGT |
| 781 | AAGATATACC ATTTCAGCAT CCAGTTAGCT GAGGTTTGAC AACACTTTTA TAGTCATTAC |
| 841 | CACAATCAAT GTATAGAATA TGTCTTTGAT AGAAAGTTCC GTTGTGTCTC TTGTATCCAC |
| 901 | TCCATGTTGC ATTTTATCCT TCTGAACTCT GTCATTGTAG ATTCATTTTG CTTTTCTTCC |
| 961 | CCTAGGGTTT CATGTACATG TAGCTAGTTA CTATAGACAC TGTCTGATCG CTTTCACTCT |
| 1021 | GCATGAATTG GAGGTTTTCT ATGTTTTTAC ATGTATCTGA AGTTATTTTT ATTGCTCAGC |
| 1081 | CATATCCAGT TGCATGCCTG GACCAATATA TGTACCTATC TGTTGATATG CTTTGAAAGT |
| 1141 | ATTTCTACTT TTTTTTTGTT TGTTTTTTGT TTTGTTTTTG AGACTATGTG GCTCTGGCTG |
| 1201 | TCCTGGAACT CGAAATATAG GCCAGGCTGG TGTGGCACTC ACAGAGACTC ACCTGCTTCT |
| 1261 | GTCTCTCACA TTCTGGGATT AAAGGTGTGG ACCATTATGC CCTGCTTATT TATTTTTTGA |
| 1321 | TGCTATGGAT AAGTCTTTAT ATGCATATAT TCCTGTGGAT ATGTTTTTAT GTCTCTCAAT |
| 1381 | AAATATGTAT TATTAACCAT ATGATTAAAG GGGCTCAAGT GTGTGTATTT TTCTCCAATA |
| 1441 | TGGCTGAGTA GAGTTTGCAT TCCCTTCAGT GTACAAGAAT GCCAGTTGAC CTTTATTCCT |
| 1501 | ATCAACACTT GGTCTTGTCT GTCTTTCACG TTCTAGCCAT ATTGACAGAC TTGTCATGGT |
| 1561 | AGCTCTTTGT AGTTTGAATT TCTGTTTCTC TGGTGTAACT TTCTTTTCAT GAAAGTTTTG |
| 1621 | TTTTCTAAGT TCATAAGGAT TTTCAACTCA GACACATTAC AGTGATACTC ACTGTTCGGC |
| 1681 | TGAAGATTTT AAAGTAGTTC ACACAGGAAG AAATGTCATA TAGCCAACAG GGGTGGAGAG |
| 1741 | GACAATAGGC CATGTTGTTC TAGGCTACAC AGCAGTTAAA TGACAAGAGT GAGCCTGCTT |
| 1801 | TCTCACCTCC AAAGTAGCGT CACCAGGCGG CATGACACTG TCATTGTCTA CAGTCAGATG |
| 1861 | ACAGGTGGAC ACAAGGGCAG AAGAGGTACA CACAGAGAGA TGCTCAGTAC ATGCATGTGC |
| 1921 | AGGGCCTGGA GGCATATCTA CTGTCTTGAT GTGTGTCATA AACCTGGCCA CTGTCCTGAT |
| 1981 | GACCATCGGC AGCTATTTGC GACAGAGTTG GTGGTTGTGC GTGTATTGTC TTCTAATGGC |
| 2041 | TTGAACAAGT AAAACATTAA TGGCAGAATG CTCTCTCCTG AGGACAGAAA GCTTGGGAAC |
| 2101 | ACAAACTGGG GACACAGCTT TGGTCCTCTG TGTACTTCTA GAAGATGCAT AGGTTGCACA |
| 2161 | AGGAAGATAG GAGGCTAGAG AGCCCGCTGC CTTCTGCAGC TGCTCATTCA TTTTGCTTTG |
| 2221 | GATTTTTTCC TTTCATTTCT CTTTCTTTCT TTCTTTCTTT CTTCCTTTCT CACCAATGGT |
| 2281 | GCTCTAGTTC TTAAGCTGTG TGCTGCAGAC ATCATCCTGG AGGCTGGTGA AACACACCTG |
| 2341 | GCCTCTCTTC CAGAGGAGCC TAGGGTCCCC TTCCAGAACT GACTTCTCTA AGGACATGGC |
| 2401 | CCCTCCTTTG AAAGTCATAC ATTAGAGCAA AGCCCTTTCC ATCCCTGCAA ATGCTGATGG |
| 2461 | CAAGGCTGGG ATAAGAACAT GGAAATGATT TCATCTGTGG GGTTCTGGGC TCAGCCTTGC |
| 2521 | AAACTAGAAT GGCAGGGGCT CATTCCTAGT AAGGAACAGA GGCAAAATAT GGAGGACAGT |
| 2581 | TATATGGAAA TGAATTGGAG CAGGTTATGA CATCTCCTTA AATGGGCATA TTTACCATCA |
| 2641 | ATAAGTTTTA TAAAACCCAC TGTCAGGTAT GGGCAATTAT CACCTCCTCT TTACAGAGGA |
| 2701 | GGAAAATGGA AGAGGCTATC TTGCCTATGC TCATGCAGCC CAGTGAGAAA GCAGGAATGA |
| 2761 | GGGCTCAGAC ATGCTAGTCA ATGGTTCTGC TCTGCTGCCT GGAGGCACCA GAATGTCCCG |
| 2821 | GCTGGGAATT CTTTATTCAC AGCAAGTTGC TTAGATGTCT GAGCTATCTA CTAAGTGGAA |
| 2881 | GTCCCGACCT TCCCTACGTC TTTGAGCTGT TGTAAAATGA ACGGAATTGA CATTATGAAG |
| 2941 | TGTTTAGGTC TGGCACGATA CAAATTCGTT ATAAACCCAT CTGCCCACCA GAGTGCTGGC |
| 3001 | AGACCGAACT TCTCCAGGGG TGGAAGCTCA GAGATGGTAC AGCACCTGAA AACATTGCAA |
| 3061 | ACCCTGGACT CTGGAGGGCG GACAACGTAG GCCCTGGGAG TGGAGGAGCC TGTCCCCTGC |
| 3121 | TCTTGCCTAC CCGGGGCCAG ACTCCAGACT CCCTGGTTCC TCACCTCCCC GCCCCCTCAC |
| 3181 | CACCCCCACC GAGGCGCTCC GAATTTCCTG CCCGACCGAG GCCCGGCTCG GGCGGGTGGA |
| 3241 | GGAGGGCTGG CATTTCCTGG CCGCCGCGTC ACTGGCTCAG CGGTGCTCGG ACAAAGCGCT |
| 3301 | GACCGACAGG CACCAGAAGC TATTTCAGGC GGCGCCCAGC TTAGCGCGCA GTTTCCGTTT |
| 3361 | TTCCACCGTC GGAAACAGGG AACAGGGAGC TTGCAGACGT CACAAACCCC CAGCCTCAGG |
| 3421 | CGTGGGTCCA GGGACCAGGA GAGGCAAGGC CCATGTGTTA GAAACAGGGT AGAGGCAGAC |
| 3481 | GCTATCCCCG CACCTTCTAT CCAACCTTAC TCCTTAACTG TCCTTGGAAA CACCAGAGAA |
| 3541 | GGCCATTTCA CACCCAGGAA AATGATCCAG TCGTCGTTGG TCAAGCCAAA TGCATAACCT |
| 3601 | TTTCAAGCCC ATAAACCTCG AGACAGCCTT ACCCCATTCC CTCTCCTGAA TTAACTAACC |
| 3661 | TGCCCCCAGA CATCCTGGAT TCTTCGATTT TCATTATTCA ACGGCGTCGT AGTTCTTCCA |
| 3721 | AACTCAGTCT TAAATACCCT GTGCGAAACA TCTACCCCAC ACCTTCTCTT CCATCTCCTG |
| 3781 | GAAGGAGAAT TAGAACAAGC TCTAACCTCT TTTCTCTGGT CACAGAACAC TTAGCCTTCA |
| 3841 | CCTCCCAGCT CCCCACACCA ACACAGCCCC TACCGCCATT TCAACCCAAG GCTTTCCTTT |
| 3901 | CCTTTCCTTT CCTTTCCTTT CCTTTCCTTT CCTTTCCTTT CCTTTCCTTT CCTTTCCTTT |
| 3961 | CCTTTCCTTT CCTTTCCTTT CCTTTTTCCT TTTTTATTAG ATATTTTCTT TATTTACATT |
| 4021 | TCAAATGTTA TCCCTTTCCT AGTTTCCCCT CCGAAAGTCC CCTATCCCTT CCCCCTCCCC |
| 4081 | CTCCCCCTGC TCCCCAACCC ACCCACTCCT GCTTCCTGGC CCTGGCATTC CCCTATACTG |
| 4141 | GGGCATAGAG CCTTCACAGG ACCAAGGGCC TCTCCTCCCA TTGATGACCA ATTAGGCCAT |
| 4201 | ACTCATGGCT CTAACTGCAT ACTGCATATG CAGTTAGAGC CATGAGTCCC ACCATGTGTT |
| 4261 | TTCTTTGATT GGTGGTTTAG TCCCAGGGAG CTCTGGGGGC ACTGGTTAGT TCATATTGTT |
| 4321 | GTTCCTCCTA TGGGGCTGCA AACCCCTTCA GCTCCTTGGA TACTTTCTCT AGTTCCTTCA |
| 4381 | TTCCAAGGGT TTTCTAAAAA AGCAAATCCG ATCTTACATA GGACAGCAAG CCCTTATGTA |
| 4441 | AACACAGTGG TAAAAACAAA ACCCTCAATT CTTCCACCCA TACTGTACCA GTTTTCTGTT |
| 4501 | TCTTACATTA ACTTTCCCCC TTTCTGTGTC AGCCCTTGGT CCAGGACGCC TGGCTTTCCT |
| 4561 | GGGAAGCACA CCCAGTTAGC TCACATACAA TATAGTTAGC CCATATAACC AAGCGAAGGC |
| 4621 | AGCACAGCTG GACTTTCATC AAATGTCACA GAGGAACAGA CAGGTGTAAC TAATACTCCA |
| 4681 | TCTCCAGTAT TGGTCCTGAA ATCTAGGAGG GGCAGAACTC AAAACAGGTG CTACTCTTTG |
| 4741 | GAATCAGCCC TTGACTGAGT CTCAGTCTGT GACCGGGTTC AGAGCTACTG GAAGGTCAAT |
| 4801 | GCAGTTTGGG ATGCTTAGTG GGGTCTATGG AATGGAAATT GAACAAGAGA GTTCAGATAG |
| 4861 | GTGCTGGCGT TACTCTAGCT ATAAGTTTGA TCAAGTTACA TCTCTTTGCC CCCTACTTTC |
| 4921 | CTTTAACATT CTTAATTTCT GTATGGCAAC CAGACAAATG CCTATGATAT CTTATTAGCT |
| 4981 | CCCTACCACC CACTTTTTAT ATTATTTCTC ATGTATATGA AACTTTGGCA TTTAAAATAT |
| 5041 | TATTATTATT CATTTAGGCT TTTTGAGAAA GGGTTTCTCT GTGTAGTCTT GGCTGTCCTG |
| 5101 | AAACTCCATA TGTAGATTAG GCTGGCCTCT AACTCAAAGG ATCTGCCTGC CTGCCTCAAT |
| 5161 | GAGAGCTGGG ATTAAAGGTA TGTGCGTTTA TTCTTTGAGT ATTTTATAAA ATGAATTTTG |
| 5221 | ATCATATTCA CCTCCTATTA CCCCTCATCT CCTCCTATCC TGATGTCTTT TTTTTTTTTT |
| 5281 | TAAATCTACA GAGAGGCTAG AGAGATGGCT CAGTGGTTAA GAGCATTGGT TGCTCTTCTG |
| 5341 | GAGGACCTGG ATTTGATTTC CAGTGTCTAT ATGGTAGCTC ACAAACTCCT GTTTCAGAGG |
| 5401 | AGCTAATGTG TCTGCCTTCT TCTAGTCTCT GGATACAGTA CATAGACATT AATACAGGCA |
| 5461 | AAACCAGGGA CCACACCCTT AAAAATGATT CCCCTTCTCA AGAAGTGTTC AACTGTCAAT |
| 5521 | AGCTCTTCAG TTAGCGGTGA AGGCTCATGA ACACCCCCCC CCCACACACA CACACACACA |
| 5581 | TACACCTACC TTCTGGCTAG AATCTTGACT GGCTTGATCC TGAGCAGGCA ACCACAGCTG |
| 5641 | TGAGTTGGTC CTTTTCTGAC CAGAAGGTAG TCACTCTGGT CTTCCTTAAC TGCTCTTACT |
| 5701 | ATCTTTTTCT TCTTTTCTTC TCTTCTGCTT CAAGGCAGGG TTTCACTATG AACCCGTGGC |
| 5761 | TGGTGGCTGA CCTGGCTCTC TATACCAGGC TGTCTTTGAA TTCATGGGTG TCCACCTCCC |
| 5821 | TCTGCCTCCC AAACACCACC ATGTGTCCAC CATGGTCTCA CTCACGTAGC CCAAACTGGC |
| 5881 | CAAGAATTCT GCTATTTTTG CCCTTATCTT TTGGATTACA ACAGCCAGTT TCCACCATCA |
| 5941 | TAGAAAGAAA AACCCAGATA CTCCTCAGCC CATTGGGTTC CTACAATGTA CCTATGGGCT |
| 6001 | TCAATGTCAA ACTTCTTTCA AATCAGGCTT CTGTCCCTCA CTAGAAATTT AATATTGAGT |
| 6061 | TATTGACACA GCCCTGTCAC CCCCTCCCCC CACTGTTTTC TCACCTATAA ATTAGGAATA |
| 6121 | ATAAAAGCAC CAATGGGGAG AAGTTGGATG AAGGGGGAAA AGTATCACTA AAGCACACTA |
| 6181 | TTATTCAAAG GTGCCCTAAT GATATCCAAT TGTATGGTAT TTAAAAAATA AAAAATAAAA |
| 6241 | GCATTACGAA ACCGGGTGTG GTGGTGCATG CCTTTAATCC CAGCACTCCA GAGGCAGAGA |
| 6301 | CAGGTGGATC TGCATCTCAA TTAGTCAGCA ACCATAGTCC CGCCCCTAAC TCCGCCCATC |
| 6361 | CCGCCCCTAA CTCCGCCCAG TTCCGCCCAT TCTCCGCCCC ATGGCTGACT AATTTTTTTT |
| 6421 | ATTTATGCAG AGGCCGAGGC CGCCTCGGCC TCTGAGCTAT TCCAGAAGTA GTGAGGAGGC |
| 6481 | TTTTTTGGAG GCCTAGGCTT TTGCAAAAAG CTCGATCCTG AGAACTTCAG GGTGAGTTTG |
| 6541 | GGGACCCTTG ATTGTTCTTT CTTTTTCGCT ATTGTAAAAT TCATGTTATA TGGAGGGGGC |
| 6601 | AAAGTTTTCA GGGTGTTGTT TAGAATGGGA AGATGTCCCT TGTATCACCA TGGACCCTCA |
| 6661 | TGATAATTTT GTTTCTTTCA CTTTCTACTC TGTTGACAAC CATTGTCTCC TCTTATTTTC |
| 6721 | TTTTCATTTT CTGTAACTTT TTCGTTAAAC TTTAGCTTGC ATTTGTAACG AATTTTTAAA |
| 6781 | TTCACTTTTG TTTATTTGTC AGATTGTAAG TACTTTCTCT AATCACTTTT TTTTCAAGGC |
| 6841 | AATCAGGGTA TATTATATTG TACTTCAGCA CAGTTTTAGA GAACAATTGT TATAATTAAA |
| 6901 | TGATAAGGTA GAATATTTCT GCATATAAAT TCTGGCTGGC GTGGAAATAT TCTTATTGGT |
| 6961 | AGAAACAACT ACATCCTGGT CATCATCCTG CCTTTCTCTT TATGGTTACA ATGATATACA |
| 7021 | CTGTTTGAGA TGAGGATAAA ATACTCTGAG TCCAAACCGG GCCCCTCTGC TAACCATGTT |
| 7081 | CATGCCTTCT TCTTTTTCCT ACAGCTCCTG GGCAACGTGC TGGTTATTGT GCTGTCTCAT |
| 7141 | CATTTTGGCA AAGAATTGTA ATACGACTCA CTATAGGGCG AATTCCACCA TGTCCAATTT |
| 7201 | ACTGACCGTA CACCAAAATT TGCCTGCATT ACCGGTCGAT GCAACGAGTG ATGAGGTTCG |
| 7261 | CAAGAACCTG ATGGACATGT TCAGGGATCG CCAGGCGTTT TCTGAGCATA CCTGGAAAAT |
| 7321 | GCTTCTGTCC GTTTGCCGGT CGTGGGCGGC ATGGTGCAAG TTGAATAACC GGAAATGGTT |
| 7381 | TCCCGCAGAA CCTGAAGATG TTCGCGATTA TCTTCTATAT CTTCAGGCGC GCGGTCTGGC |
| 7441 | AGTAAAAACT ATCCAGCAAC ATTTGGGCCA GCTAAACATG CTTCATCGTC GGTCCGGGCT |
| 7501 | GCCACGACCA AGTGACAGCA ATGCTGTTTC ACTGGTTATG CGGCGGATCC GAAAAGAAAA |
| 7561 | CGTTGATGCC GGTGAACGTG CAAAACAGGC TCTAGCGTTC GAACGCACTG ATTTCGACCA |
| 7621 | GGTTCGTTCA CTCATGGAAA ATAGCGATCG CTGCCAGGAT ATACGTAATC TGGCATTTCT |
| 7681 | GGGGATTGCT TATAACACCC TGTTACGTAT AGCCGAAATT GCCAGGATCA GGGTTAAAGA |
| 7741 | TATCTCACGT ACTGACGGTG GGAGAATGTT AATCCATATT GGCAGAACGA AAACGCTGGT |
| 7801 | TAGCACCGCA GGTGTAGAGA AGGCACTTAG CCTGGGGGTA ACTAAACTGG TCGAGCGATG |
| 7861 | GATTTCCGTC TCTGGTGTAG CTGATGATCC GAATAACTAC CTGTTTTGCC GGGTCAGAAA |
| 7921 | AAATGGTGTT GCCGCGCCAT CTGCCACCAG CCAGCTATCA ACTCGCGCCC TGGAAGGGAT |
| 7981 | TTTTGAAGCA ACTCATCGAT TGATTTACGG CGCTAAGGAT GACTCTGGTC AGAGATACCT |
| 8041 | GGCCTGGTCT GGACACAGTG CCCGTGTCGG AGCCGCGCGA GATATGGCCC GCGCTGGAGT |
| 8101 | TTCAATACCG GAGATCATGC AAGCTGGTGG CTGGACCAAT GTAAATATTG TCATGAACTA |
| 8161 | TATCCGTAAC CTGGATAGTG AAACAGGGGC AATGGTGCGC CTGCTGGAAG ATGGCGATCT |
| 8221 | CGAGCCATCT GCTGGAGACA TGAGAGCTGC CAACCTTTGG CCAAGCCCGC TCATGATCAA |
| 8281 | ACGCTCTAAG AAGAACAGCC TGGCCTTGTC CCTGACGGCC GACCAGATGG TCAGTGCCTT |
| 8341 | GTTGGATGCT GAGCCCCCCA TACTCTATTC CGAGTATGAT CCTACCAGAC CCTTCAGTGA |
| 8401 | AGCTTCGATG ATGGGCTTAC TGACCAACCT GGCAGACAGG GAGCTGGTTC ACATGATCAA |
| 8461 | CTGGGCGAAG AGGGTGCCAG GCTTTGTGGA TTTGACCCTC CATGATCAGG TCCACCTTCT |
| 8521 | AGAATGTGCC TGGCTAGAGA TCCTGATGAT TGGTCTCGTC TGGCGCTCCA TGGAGCACCC |
| 8581 | AGGGAAGCTA CTGTTTGCTC CTAACTTGCT CTTGGACAGG AACCAGGGAA AATGTGTAGA |
| 8641 | GGGCATGGTG GAGATCTTCG ACATGCTGCT GGCTACATCA TCTCGGTTCC GCATGATGAA |
| 8701 | TCTGCAGGGA GAGGAGTTTG TGTGCCTCAA ATCTATTATT TTGCTTAATT CTGGAGTGTA |
| 8761 | CACATTTCTG TCCAGCACCC TGAAGTCTCT GGAAGAGAAG GACCATATCC ACCGAGTCCT |
| 8821 | GGACAAGATC ACAGACACTT TGATCCACCT GATGGCCAAG GCAGGCCTGA CCCTGCAGCA |
| 8881 | GCAGCACCAG CGGCTGGCCC AGCTCCTCCT CATCCTCTCC CACATCAGGC ACATGAGTAA |
| 8941 | CAAAGGCATG GAGCATCTGT ACAGCATGAA GTGCAAGAAC GTGGTGCCCC TCTATGACCT |
| 9001 | GCTGCTGGAG ATGCTGGACG CCCACCGCCT ACATGCGCCC ACTAGCCGTG GAGGGGCATC |
| 9061 | CGTGGAGGAG ACGGACCAAA GCCACTTGGC CACTGCGGGC TCTACTTCAT CGCATTCCTT |
| 9121 | GCAAAAGTAT TACATCACGG GGGAGGCAGA GGGTTTCCCT GCCACAGTCT GAGAGCTCCC |
| 9181 | TGGCGGAATT CGGATCTTAT TAAAGCAGAA CTTGTTTATT GCAGCTTATA ATGGTTACAA |
| 9241 | ATAAAGCAAT AGCATCACAA ATTTCACAAA TAAAGCATTT TTTTCACTGC ATTCTAGTTG |
| 9301 | TGGTTTGTCC AAACTCATCA ATGTATCTTA TCATGTCTGG TCGACCGATG CCCTTGAGAG |
| 9361 | CCTTCAACCC AGTCAGCTCC TTCCGGTGGG CGCGGGGCAT GACTATCGTC GCCGCACTTA |
| 9421 | TGACTGTCTT CTTTATCATG CAACTCGTAG GACAGGTGCC GGCAGCGCTC TTCCGCTTCC |
| 9481 | TCGCTCACTG ACTCGCTGCG CTCGGTCGTT CGGCTGCGGC GAGCGGTATC AGCTCACTCA |
| 9541 | AAGGCGGTAA TACGGTTATC CACAGAATCA GGGGATAACG CAGGAAAGAA CATGTGAGCA |
| 9601 | AAAGGCCAGC AAAAGGCCAG GAACCGTAAA AAGGCCGCGT TGCTGGCGTT TTTCCATAGG |
| 9661 | CTCCGCCCCC CTGACGAGCA TCACAAAAAT CGACGCTCAA GTCAGAGGTG GCGAAACCCG |
| 9721 | ACAGGACTAT AAAGATACCA GGCGTTTCCC CCTGGAAGCT CCCTCGTGCG CTCTCCTGTT |
| 9781 | CCGACCCTGC CGCTTACCGG ATACCTGTCC GCCTTTCTCC CTTCGGGAAG CGTGGCGCTT |
| 9841 | TCTCATAGCT CACGCTGTAG GTATCTCAGT TCGGTGTAGG TCGTTCGCTC CAAGCTGGGC |
| 9901 | TGTGTGCACG AACCCCCCGT TCAGCCCGAC CGCTGCGCCT TATCCGGTAA CTATCGTCTT |
| 9961 | GAGTCCAACC CGGTAAGACA CGACTTATCG CCACTGGCAG CAGCCACTGG TAACAGGATT |
| 10021 | AGCAGAGCGA GGTATGTAGG CGGTGCTACA GAGTTCTTGA AGTGGTGGCC TAACTACGGC |
| 10081 | TACACTAGAA GAACAGTATT TGGTATCTGC GCTCTGCTGA AGCCAGTTAC CTTCGGAAAA |
| 10141 | AGAGTTGGTA GCTCTTGATC CGGCAAACAA ACCACCGCTG GTAGCGGTGG TTTTTTTGTT |
| 10201 | TGCAAGCAGC AGATTACGCG CAGAAAAAAA GGATCTCAAG AAGATCCTTT GATCTTTTCT |
| 10261 | ACGGGGTCTG ACGCTCAGTG GAACGAAAAC TCACGTTAAG GGATTTTGGT CATGAGATTA |
| 10321 | TCAAAAAGGA TCTTCACCTA GATCCTTTTA AATTAAAAAT GAAGTTTTAA ATCAATCTAA |
| 10381 | AGTATATATG AGTAAACTTG GTCTGACAGT TACCAATGCT TAATCAGTGA GGCACCTATC |
| 10441 | TCAGCGATCT GTCTATTTCG TTCATCCATA GTTGCCTGAC TCCCCGTCGT GTAGATAACT |
| 10501 | ACGATACGGG AGGGCTTACC ATCTGGCCCC AGTGCTGCAA TGATACCGCG AGACCCACGC |
| 10561 | TCACCGGCTC CAGATTTATC AGCAATAAAC CAGCCAGCCG GAAGGGCCGA GCGCAGAAGT |
| 10621 | GGTCCTGCAA CTTTATCCGC CTCCATCCAG TCTATTAATT GTTGCCGGGA AGCTAGAGTA |
| 10681 | AGTAGTTCGC CAGTTAATAG TTTGCGCAAC GTTGTTGCCA TTGCTACAGG CATCGTGGTG |
| 10741 | TCACGCTCGT CGTTTGGTAT GGCTTCATTC AGCTCCGGTT CCCAACGATC AAGGCGAGTT |
| 10801 | ACATGATCCC CCATGTTGTG CAAAAAAGCG GTTAGCTCCT TCGGTCCTCC GATCGTTGTC |
| 10861 | AGAAGTAAGT TGGCCGCAGT GTTATCACTC ATGGTTATGG CAGCACTGCA TAATTCTCTT |
| 10921 | ACTGTCATGC CATCCGTAAG ATGCTTTTCT GTGACTGGTG AGTACTCAAC CAAGTCATTC |
| 10981 | TGAGAATAGT GTATGCGGCG ACCGAGTTGC TCTTGCCCGG CGTCAATACG GGATAATACC |
| 11041 | GCGCCACATA GCAGAACTTT AAAAGTGCTC ATCATTGGAA AACGTTCTTC GGGGCGAAAA |
| 11101 | CTCTCAAGGA TCTTACCGCT GTTGAGATCC AGTTCGATGT AACCCACTCG TGCACCCAAC |
| 11161 | TGATCTTCAG CATCTTTTAC TTTCACCAGC GTTTCTGGGT GAGCAAAAAC AGGAAGGCAA |
| 11221 | AATGCCGCAA AAAAGGGAAT AAGGGCGACA CGGAAATGTT GAATACTCAT ACTCTTCCTT |
| 11281 | TTTCAATATT ATTGAAGCAT TTATCAGGGT TATTGTCTCA TGAGCGGATA CATATTTGAA |
| 11341 | TGTATTTAGA AAAATAAACA AATAGGGGTT CCGCGCACAT TTCCCCGAAA AGTGCCACCT |
| 11401 | GACGCGCCCT GTAGCGGCGC ATTAAGCGCG GCGGGTGTGG TGGTTACGCG CAGCGTGACC |
| 11461 | GCTACACTTG CCAGCGCCCT AGCGCCCGCT CCTTTCGCTT TCTTCCCTTC CTTTCTCGCC |
| 11521 | ACGTTCGCCG GCTTTCCCCG TCAAGCTCTA AATCGGGGGC TCCCTTTAGG GTTCCGATTT |
| 11581 | AGTGCTTTAC GGCACCTCGA CCCCAAAAAA CTTGATTAGG GTGATGGTTC ACGTAGTGGG |
| 11641 | CCATCGCCCT GATAGACGGT TTTTCGCCCT TTGACGTTGG AGTCCACGTT CTTTAATAGT |
| 11701 | GGACTCTTGT TCCAAACTGG AACAACACTC AACCCTATCT CGGTCTATTC TTTTGATTTA |
| 11761 | TAAGGGATTT TGCCGATTTC GGCCTATTGG TTAAAAAATG AGCTGATTTA ACAAAAATTT |
| 11821 | AACGCGAATT TTAACAAAAT ATTAACGCTT ACAATTTGCC ATTCGCCATT CAGGCTGCGC |
| 11881 | AACTGTTGGG AAGGGCGATC GGTGCGGGCC TCTTCGCTAT TACGCCAGCC CAAGCTACCA |
| 11941 | TGATAAGTAA GTAATATTAA GGTACGTGGA GGTTTTACTT GCTTTAAAAA CCTCCCACAC |
| 12001 | CTCCCCCTGA ACCTGAAACA TAAAATGAAT GCAATTGTTG TTGTTAACTT GTTTATTGCA |
| 12061 | GCTTATAATG GTTACAAATA AAGCAATAGC ATCACAAATT TCACAAATAA AGCATTTTTT |
| 12121 | TCACTGCATT CTAGTTGTGG TTTGTCCAAA CTCATCAATG TATCTTATGG TACTGTAACT |
| 12181 | GAGCTAACAT AA |
| // | |

We claim:

1. A method of producing a transgenic animal comprising the steps of:
providing a transgenic cell line which conditionally expresses a compromiser gene corresponding to a predetermined lineage complementary to a target lineage;
providing a donor embryo having a specific gene deficiency corresponding to the target lineage or which conditionally expresses a compromiser gene corresponding to the target lineage;
introducing the cell line into the donor embryo; and
activating the compromiser gene(s) at a predetermined time in the development of the donor embryo so that only the target lineage of the transgenic cell line survives and only the complementary lineage of the embryo survives.

2. The method of claim 1, wherein the transgenic cell line is embryonic cells, embryonic stem cells, precursor or induced pluripotent stem cells [EC/ES/P/iPS cells].

3. The method of claim 1, wherein the target lineage corresponds to the hematopoietic and endothelial system of the transgenic animal.

4. The method of claim 1, wherein the target lineage corresponds to an organ of the transgenic animal.

5. The method of claim 1, wherein the target lineage corresponds to tissue of the transgenic animal.

6. The method of claim 1, wherein the transgenic cell line is human.

7. The method of claim 6, wherein the donor embryo is a non-human animal.

8. The method of claim 7, wherein the non-human animal is mouse or pig.

9. The method of claim 1, wherein the donor embryo is a morula-stage embryo.

10. The method of claim 1, wherein the introducing step is in vivo.

11. The method of claim 1, wherein the introducing step is in vitro.

12. The method of claim 1, wherein the compromiser gene is selected from Diphtheria Toxin A (DT A), Herpes Simplex Virus-Thymidine Kinase (HSV-TK) or hypoxanthine phosphoribosyltransferase (hprt).

13. The method of claim 1, wherein the activating step includes a recombination control drug introduced into the host embryo.

14. A method of producing a transgenic animal comprising the steps of:
providing a transgenic cell line which conditionally expresses a compromiser gene corresponding to a predetermined lineage complementary to a target lineage;
providing a donor embryo having a specific gene deficiency corresponding to the target lineage or a donor embryo which conditionally expresses a compromiser gene corresponding to the target lineage;
introducing the transgenic cell line into the donor embryo; and
activating the compromiser gene(s) at a predetermined time in the growth of the donor embryo so that only the differentiated cells of the target lineage of the transgenic cell line will survive and only the differentiated cells of the complementary lineage of the embryo will survive.

15. A method of directing the development of an embryo comprising the steps of:
providing a transgenic cell line which conditionally expresses a compromiser gene corresponding to a predetermined lineage;
introducing the cell line into a donor embryo having a specific gene deficiency or a compromiser gene corresponding to a complementary lineage; and
activating the compromiser gene(s) at a predetermined time in the growth of the donor embryo so that the complementary lineage of the transgenic cell line will substitute for the complementary lineage of the donor embryo as the embryo develops.

16. A chimeric animal comprising:
a target tissue and/or organ differentiated from the genotype of a transgenic cell line; and
all remaining non-target tissues and/or organs differentiated from the genotype of a donor embryo.

17. The chimeric animal of claim 16, wherein the transgenic cell line is embryonic cells, embryonic stem cells, precursor or induced pluripotent stem cells [EC/ES/P/iPS cells].

18. The chimeric animal of claim 16, wherein the transgenic cell line is human.

19. The chimeric animal of claim 17, wherein the donor embryo is a non-human animal.

20. The method of claim 19, wherein the non-human animal is mouse or pig.
